(19) United States
(12) Patent Application Publication
(10) Pub. No.: US 2023/0372292 A1
(43) Pub. Date: Nov. 23, 2023

(54) TREATMENTS OF CORONAVIRUS INFECTIONS, CYTOKINE RELEASE SYNDROME, CYTOKINE STORM SYNDROME, OR DISEASES ASSOCIATED WITH EXCESSIVE ACTIVATION OF INFLAMMASOMES BY THE USE OF INHIBITORS OF INFLAMMATORY CASPASES

(71) Applicant: INSTITUT NATIONAL DE LA SANTE ET DE LA RECHERCHE MEDICALE, Paris (FR)

(72) Inventors: Etienne JACOTOT, Paris (FR); Bernadette BUNG, MARLY-LE-ROI (FR); Newton HOWARD, Washington, DC (US)

(21) Appl. No.: 18/247,889

(22) PCT Filed: Oct. 7, 2021
(86) PCT No.: PCT/EP2021/077727

§ 371 Date: Apr. 5, 2023

Related U.S. Application Data
(60) Provisional application No. 63/088,652, filed on Oct. 7, 2020.
(60) Provisional application No. 63/160,768, filed on Mar. 13, 2021.

(30) Foreign Application Priority Data
May 12, 2021 (FR) FR2105065
May 19, 2021 (EP) 21174850.4

(51) Int. Cl. A61K 31/4025; A61K 45/06; A61P 31/14

(52) U.S. Cl. CPC A61K 31/4025; A61K 45/06; A61P 31/14

(57) ABSTRACT

The present invention relates to methods to inhibit the inflammatory caspases for ameliorating, preventing, or treating diseases associated with excessive inflammatory responses to pathogenic infection or danger signalization including coronaviruses infection and inflammatory consequences of coronaviruses infection in humans, SARS, MERS, COVID-19. The identification of a central role of the inflammatory Caspases in the pathogenic activity of the canonic and non-canonic inflammasomes provide a general method to treat acute diseases including SARS, MERS, COVID-19, cytokine release syndrome, cytokine storm syndrome, pyroptosis and inflammasome-related multi-organ failure, and pathogen-induced acute respiratory distress syndromes. Compounds to be repurposed and new pharmaceutical compositions are claimed.

### FIELD OF THE INVENTION

The present invention relates to methods to inhibit the inflammatory caspases subfamilly for ameliorating, preventing, or treating diseases associated with excessive inflammatory responses to pathogenic infection or danger signalisation including coronaviruses infection and inflammatory consequences of coronaviruses infection in humans.

### BACKGROUND OF THE INVENTION

Caspases (cysteine-dependent aspartate-specific proteases) are a family of cysteine endoproteases (C14A family of the CD clan), unique to the animal kingdom, which cleave peptides and proteins specifically after an Aspartate residue [Handbook of Proteolytic Enzymes 2013 (Edited by Neil D. Rawlings & Guy Salvesen), Vol. 2 (chap. 505-513):2237-2285]. This recognition specificity is rare among proteases and unique among cysteine proteases. Caspases are central effectors and regulators of apoptosis and inflammation [Van Opdenbosch N, Lamkanfi M. Immunity. Caspases in Cell Death, Inflammation, and Disease. 2019; 50(6):1352-1364]. They are also involved in the regulation of non-apoptotic cell death pathways as well as in various physiological processes which include proliferation, differentiation, cell migrations, several fine functions of the developing and mature nervous system (synaptic plasticity, axonal guidance, LTP, pruning of dendritic spines), cell cycle control and stress responses [Shalini S, Dorstyn L, Dawar S, Kumar S. Old, new and emerging functions of caspases. Cell Death Differ. 2015; 22:526-39]. The Caspase family has been well characterized in terms of gene expression, structure, activation and catalytic activity [Pop C, Salvesen GS. Human caspases: activation, specificity, and regulation. J Biol Chem. 2009, 284(33):21777-81].

All caspases contain two subunits (p20 and p10) which form the catalytic domain. They are synthesized as zymogens (enzymes awaiting activation). The caspases initiating apoptosis (Caspase-2, -8, -9, -10) and inflammatory (Caspase-1, -4, -5, -11, -12) possess N-terminal prodomains which determine their dimerization and/or their homotypic interaction with adapter proteins. We distinguish between DED domains (Death Effector Domain) which are found in tandem in Caspases -8 and -10, CARD domains (Caspase Activation and Recrutement Domain) present individually in all inflammatory Caspases as well as in Caspases -2 and -9. These DED or CARD domains are used for the recruitment of zymogens into activation platforms. Apoptosis-executing Caspases (Caspase-3, -6, -7) have a short N-terminal pro-domain lacking a recruiting domain [Shalini S, Dorstyn L, Dawar S, Kumar S. Old, new and emerging functions of caspases. Cell Death Differ. 2015; 22:526-39]. To be activated, the executing Caspases must be cleaved at their catalytic domain. A series of cleavage events, by initiating Caspases, first separate the large and small subunits and then eliminate the prodomain. This cleavage is dispensable for the activation of the initiator and inflammatory caspases. Caspases have a broader spectrum of implication in regulated cell death than initially believed. Among the best described regulated cell death pathways are: apoptosis, death associated with autophagy, and regulated necrosis including pyroptosis (inflammatory death) and necroptosis (which involves the Ripoptosome RIP1K/RIP3K) and the MLKL (mixed lineage kinase domain-like) pseudo-kinase. All these death pathways are regulated by several individual Caspases.

The apoptosis-initiating caspases and the inflammatory caspases are activated by their recruitment within specialized platforms. Five activation platforms have been described: the canonical inflammasomes for Caspase-1, the Piddosome for Caspase-2; bacterial lipopolysaccharides (LPS complexes) for caspases-4, -5, and -11, the DISC for Caspase-8, and the apoptosome for Caspase-9.

Inflammasomes are multiprotein signaling platforms that control the inflammatory response and coordinate antimicrobial defenses. They assemble when a PRR (Pattern Recognition Receptor) type receptor detects a molecular pattern of a pathogenic microorganism (PAMP) or an endogenous danger signal (DAMP) in the cytosol of the host cell and activate inflammatory caspases to produce the maturation of the cytokines interleukin-1β (IL1 (3) and IL-18 and induce pyroptosis, a form of regulated necrotic cell death (from the Greek pyros: fire or fever; ptosis: fall). Five sensor-receptors capable of assembling each, according to the type of signal received, a canonical inflammasome have been well described: NLRP1 (NOD-like receptor (NLR) family, pyrin domain-containing protein 1), NLRP3, NLRC4 (NOD-, leucin-rich repeat (LRR)—and CARD-containing 4), AIM2 (absent in melanoma 2), and Pyrin. During their activation, these receptors recruit, through homotypic PYD-PYD or CARD-CARD interactions, the bipartite adapter protein ASC (Apoptosis-associated speck-like protein containing a CARD), which contains a Pyrin domain (PYD) and a domain CARD. The ASC CARD domain is required to recruit pro-Caspase-1 into the multimeric complex [He Y, Hara H, Núñez G. Mechanism and Regulation of NLRP3 Inflammasome Activation. Trends Biochem Sci. 2016; 41(12):1012-1021]. In the case of the NLRP3-ASC-Casp1 inflammasome, it was recently discovered that the platform must recruit the serine-threonine kinase NEK7 (NIMA-related kinase 7) to activate properly [He Y, Zeng M Y, Yang D, et al., NEK7 is an essential mediator of NLRP3 activation downstream of potassium efflux. Nature. 2016; 18; 530(7590):354-7]. Within the canonical inflammasome, Caspase-1 activates in a proximity-induced self-proteolytic mode. This is followed by maturation by cleavage of pro-IL1β and pro-IL18 as well as the cleavage of Gasdermin D (GSDMD). The N-terminal fragment of GSDMD is the final effector of pyroptosis via pore formation in membranes and allows the release of mature IL 1β and IL 18 [Liu X, Zhang Z, Ruan J, et al., Inflammasome-activated gasdermin D causes pyroptosis by forming membrane pores. Nature. 2016; 535(7610):153-8] [Shi J, Zhao Y, Wang K, et al., Cleavage of GSDMD by inflammatory caspases determines pyroptotic cell death. Nature. 2015; 526(7575):660-5].

Non canonical inflammasomes: LPS is the main endotoxin of gram-negative bacteria and one of the strongest activators of the immune system. It is made up of three domains: 0 antigen (a repeating glycan polymer), Core (an oligosaccharide component, usually heptose and ulosonic acid), and Lipid A (a phosphorylated disaccharide of glucosamine decorated with multiple fatty acids). The latter is very conserved and responsible for the toxicity of LPS. The LPS receptor on the cell surface, was found to be TLR4 (Toll-Like Receptor 4), a discovery that was rewarded in 2011 by the Nobel Prize in physiology or Medicine [Poltorak A, He X, Smirnova I, et al., Defective LPS signaling in C3H/HeJ and C57BL/10ScCr mice: Mutations in TIr4 gene. Science 1998; 282:2085-2088]. Several years later, the demonstration of the direct activation of Caspase-11 (in mice) and Caspases-4/5 (in humans) by intracellular LPS was an unexpected and major discovery [Kayagaki N, Warming S, Lamkanfi S, et al., Non-canonical inflammasome activation targets caspase-11. Nature 2011; 479 :117-122] [Kayagaki N, Wong M T, Stowe I B, et al., Noncanonical inflammasome activation by intracellular LPS independent of TLR4. Science. 2013; 341(6151):1246-9] [Shi J, Zhao Y, Wang Y, et al., Inflammatory caspases are innate immune receptors for intracellular LPS. Nature. 2014; 514(7521):187-92] [Rathinam V A K, Zhao Y, Shao F. Innate immunity to intracellular LPS. Nat Immunol. 2019; 20(5):527-533]. LPS is carried into the cell by bacterial AB5-type toxins such as cholera toxin. Lipid A of LPS binds directly to the CARD domain of Caspase-11, which is sufficient to trigger its oligomerization and catalytic activity. The effect is similar on human orthologs Caspases-4 and -5, with higher affinity. Thus, Caspases-4, -5, -11 do not need to interact with an NLR to be activated and are rather cytosolic receptors for LPS. Once activated by LPS, Caspases-4/5/11, directly cleave Gasdermin D which triggers the insertion of its N-terminal domain into the plasma membrane which then leads to the formation of a pore (18 nm internal diameter) and cell death by pyroptosis [Aglietti R A, Estevez A, Gupta A, et al., GsdmD p30 elicited by caspase-11 during pyroptosis forms pores in membranes. Proc Natl Acad Sci U S A. 2016; 113(28):7858-63] [Ding J, Wang K, Liu W, et al., Pore-forming activity and structural autoinhibition of the gasdermin family. Nature. 2016; 535(7610):111-6]. Thus pyroptosis can be redefined as a programmed necrosis performed by Gasdermin D [Kayagaki N, Stowe I B, Lee B L et al., Caspase-11 cleaves gasdermin D for non-canonical inflammasome signalling. Nature. 2015; 526(7575):666-71]. This mechanism is operational in most cell types. In the case of monocytes / macrophages and dendritic cells, Gasdermin D pore formation can induce the activation of the NLRP3-ASC-proCaspl inflammasome for the processing of cytokines IL-1β and IL-18. These interleukins can then use the Gasdermin D pores as a conduit out of the cells. This combination of canonical and non-canonical inflammasome action in response to cytosolic LPS is critical for antibacterial defense and septic shock. Pyroptosis occurs, for example, in response to invasive Gram-negative infections, such as Salmonella and Shigella [Baker P J, Boucher D, Bierschenk D, et al., NLRP3 inflammasome activation downstream of cytoplasmic LPS recognition by both caspase-4 and caspase-5. Eur J Immunol. 2015; 45(10):2918-26] [Suzuki T, Franchi L, Toma C, et al., Differential regulation of caspase-1 activation, pyroptosis, and autophagy via Ipaf and ASC in Shigella-infected macrophages. PLoS Pathog. 2007; 3(8):e111]. In addition, the human non-canonical inflammasome detects a greater diversity of LPS than its murine counterpart [Lagrange B, Benaoudia S, Wallet P, et al., Human caspase-4 detects tetra-acylated LPS and cytosolic Francisella and functions differently from murine caspase-11. Nat Commun. 2018; 9(1):242].

Pyroptosis of macrophages that have phagocytosed viruses rapidly releases a myriad of a a mins, including viral particles, cytokines, chemokines, LDH, ATP, and ROS, prompting an immediate reaction from surrounding immune cells and thus inducing a pyroptotic chain reaction. Moreover, pyroptosis would allow viral Ags and RNA to be disseminated in the circulation and possibly generate immune complex and deposition in target organs, such as kidney, to initiate severe inflammatory cascade.

Caspases Inhibitors and Inflammatory Caspases Inhibitors.

Caspases cleave peptides and proteins after an Asp (P1 position) [Handbook of Proteolytic Enzymes 2013 (Edited by Neil D. Rawlings & Guy Salvesen), Vol.2 (chap. 505-513) : 2237-85]. This unique property among cysteine-proteases, has led to the development of hundreds of active site-directed small peptides and peptidomimetics that are Caspase-specific [Poreba M, Szalek A, Kasperkiewicz P, Rut W, Salvesen GS , Drag M. Small Molecule Active Site Directed Tools for Studying Human Caspases. Chem Rev. 2015, 115(22):12546-629]. Indeed, medicinal chemistry of Caspase inhibitors led to potent druggable peptide derivatives (e.g., Q-VD-OPh, an irreversible pan-caspase inhibitor [Chauvier D, Ankri S, Charriaut-Marlangue C, Casimir R, Jacotot E. Broad-spectrum caspase inhibitors: from myth to reality? Cell Death Differ. 2007 February; 14(2):387-91]), to the clinical development of potent peptidomimetics (e.g., Belnacasan/VX765 [Wannamaker W, Davies R, Namchuk M, Pollard J, Ford P, Ku G, Decker C, Charifson P, Weber P, Germann U A, Kuida K, Randle J C (May 2007). “(S)-1-((S)-2-{[1-(4-amino-3-chloro-phenyl)-methanoyl]-amino}-3,3-dimethyl-butanoyl)-pyrrolidine-2-carboxylic acid ((2R,3S)-2-ethoxy-5-oxo-tetrahydro-furan-3-yl)-amide (VX-765), an orally available selective interleukin (IL)-converting enzyme/caspase-1 inhibitor, exhibits potent anti-inflammatory activities by inhibiting the release of IL-1beta and IL-18”. The Journal of Pharmacology and Experimental Therapeutics. 321 (2): 509-16], a reversible inhibitor of inflammatory caspases), and to the advanced clinical development of safe broad-spectrum caspase inhibitor (e.g., Emricasan [Steven D Linton, Teresa Aja, Robert A Armstrong, Xu Bai, Long-Shiuh Chen, Ning Chen, Brett Ching, et el., First-in-class pan caspase inhibitor developed for the treatment of liver disease. J Med Chem. 2005, 48(22):6779-82], an irreversible pan-caspase inhibitor) [Hong-Rae Kim, Ravichandra Tagirasa, and Euna Yoo*. Covalent Small Molecule Immunomodulators Targeting the Protease Active Site. J. Med. Chem. 2021, 64, 9, 5291-5322]. However, the design of active site-directed inhibitors selective of one given individual Caspase is highly challenging because caspases have similar active sites, and most Asp^{P1}-containing small peptides are efficiently recognized by several caspases [Poreba M, Strózyk A, Salvesen G S, Drag M. Caspase substrates and inhibitors. Cold Spring Harb Perspect Biol. 2013, 5(8):a008680]. As examples of inflammatory caspases inhibitors, reference is made to the inflammatory caspases inhibitors cited in Linton S D, Aja T, Armstrong R A, et al., First-in-class pan caspase inhibitor developed for the treatment of liver disease. J Med Chem. 2005; 48(22):6779-82, or in Garcia-Tsao G, Bosch J, Kayali Z, et al., Randomized Placebo-Controlled Trial of Emricasan in Non-alcoholic Steatohepatitis (NASH) Cirrhosis with Severe Portal Hypertension. J Hepatol. 2019 Dec 20. pii: S0168-8278(19)30724, or in Harrison S A, Goodman Z, Jabbar A, et al., A randomized, placebo-controlled trial of emricasan in patients with NASH and F1-F3 fibrosis. J Hepatol. 2019 Dec 27. pii: S0168-8278(19)30758-5.

The compound (S)-1-((S)-2-((1-(4-Amino-3-chloro-phenyl)-methanoyl)-amino)-3,3-dimethyl-butanoyl)-pyrrolidine-2-carboxylic acid ((2R,3S)-2-ethoxy-5-oxo-tetrahydro-furan-3-yl)-amide, (S)-1-((S)-2-(4-Amino-3-chlorobenzamido)-3,3-dimethylbutanoyl)-N-((2R,3S)-2-ethoxy-5-oxotetrahydrofuran-3-yl)pyrrolidine-2-carboxamide, (S)-1-((S)-2-(4-Amino-3-chlorobenzamido)-3,3-dimethylbutanoyl)-N-((2R,3S)-2-ethoxy-5-oxotetrahydrofuran-3-yl)pyrrolidi ne-2-ca rboxa mide, also known as VX-765 or Belnacasan, is an inhibitor of the inflammatory Caspases including Caspase-1, human Caspase-4, human Caspase-5, and mouse Caspase-11. It has been developed to inhibit preferentially Caspase-1. (Boxer M B, Shen M, Auld D S, Wells J A, Thomas C J. A small molecule inhibitor of Caspase 1. Reports from the NIH Molecular Libraries Program [Internet]. Bethesda (MD): National Center for Biotechnology Information (US); 2010-.2010 Feb. 25 [updated 2011 Mar. 3]); (Wannamaker W, Davies R, Namchuk M, Pollard J, Ford P, Ku G, Decker C, Charifson P, Weber P, Germann U A, Kuida K, Randle J C. (S)-1-((S)-2-{[1-(4-amino-3-chloro-phenyl)-methanoyl]-amino}-3,3-dimethyl-butanoyl)-pyrrolidine-2-carboxylic acid ((2 R,3S)-2-ethoxy-5-oxo-tetrahydro-furan-3-yl)-amide (VX-765), an orally available selective interleukin (IL)-converting enzyme/caspase-1 inhibitor, exhibits potent anti-inflammatory activities by inhibiting the release of IL-lbeta and IL-18. J Pharmacol Exp Ther. 2007; 321(2):509-16).

Belnacasan is an acetyl pro-drug with a reversible leaving group (aldehyde). A cell-permeable prodrug. Under the action of esterases, its active metabolite is produced (VRT-043198) [54]. Belnacasan, upon conversion in its active form VRT-043198 is a potent inhibitor of Caspase 1 (Ki=0.8 nm) and Caspase-4 (Ki<0.6 nM) which exhibits a more moderate effect on Caspase-8 (Ki=100 nM), weak on Caspase-9 (Ki=1 μM), and very low on Caspase-3 (Ki=16 μM) and Caspase-7 (Ki=21.5 μM). Belnacasan /VX-765 structure:

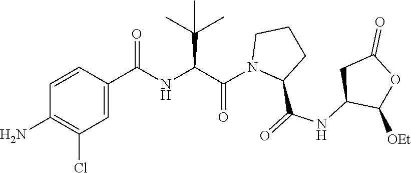 [SMILES: CCO[C@@H]1OC(=O)C[C@@H]1NC(=O)[C@@H]1CCCN1C(=O)[C@@H](NC(=O)c1ccc(N)c(Cl)c1)C(C)(C)C]

VRT-043198 (Belnacasan active metabolite) structure, that exists as a mixture of two isoforms:

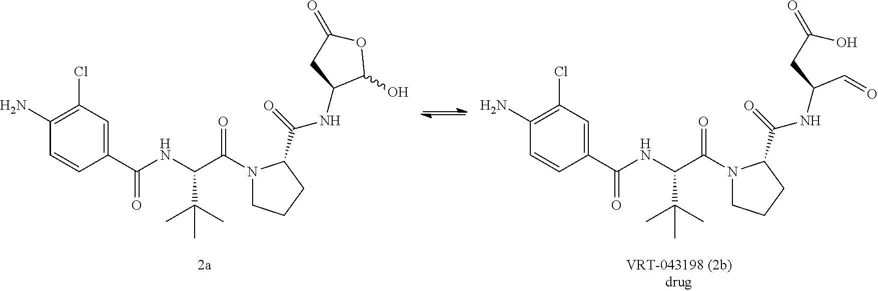

Belnacasan has been shown to be effective in cellular models of interleukin-1β release after LPS treatment. It has shown significant efficacy in animal models of osteoarthritis, but also epilepsy, and myocardial infarction (in combination with a platelet inhibitor). Belnacasan is orally active and well tolerated in humans. During a clinical phase 2b in 60 patients with epilepsy (12 placebos, 48 VX-765), the primary endpoint of safety and tolerance was reached. The secondary endpoint (decrease in the frequency of epileptic episodes) gave a favorable trend (15.6% reduction in the VX-765 group vs.

8.3% in the placebo group) but no statistical superiority. Another phase 2 study with Belnacasan in patients with psoriasis has been performed, but the results were not disclosed. More recently, preclinical efficacy data suggested that Belnacasan could be evaluated in HIV-1 seropositive patients to reduce pyroptosis of CD4 +lymphocytes, as well as for the treatment of Alzheimer's disease and other neurodegenerative diseases such as multiple sclerosis.

Belnacasan as compound and pharmaceutical preparation was patented by Vertex (EP1396492; U.S. Pat. No. 7,417,029; WO2005117846A2). However, its use in the treatment of coronavirus-induced disease and coronavirus-induced inflammatory response has not been claimed.

The use of inflammatory Caspases inhibitors, targeting Caspase-1, Caspase-4 and Caspase-5 has not been proposed previously for the treatment of Coronaviruses-mediated disease including SARS, MERS and COVID-19, and their complications that include diffuse alveolar damage, acute respiratory distress syndrome with a distinct time course, imaging and laboratory features, thromboembolic phenomena, and multi organ failure.

Coronaviruses

The severe acute respiratory syndrome coronavirus 2 (SARS-CoV-2), an enveloped single-stranded RNA virus from the Coronavirus (CoV) family. CoVs are widely distributed among mammals and birds. They cause mainly respiratory or enteric diseases, and in some cases neurological or hepatic pathologies. In humans, the so-called common CoVs cause colds and upper respiratory tract conditions that are mild in immunocompetent individuals. However, in immunologically weakened or fragile people, and in the case of certain variant CoVs, they can infect the deep airways and trigger pneumonia, worsen asthma and/or trigger different types of acute respiratory syndromes (Paul S. Masters.The Molecular Biology of Coronaviruses. Advances in Virus Research 2006, 66:193-292). The 3 coronaviruses recently appeared in human populations SARS-CoV (in 2003), MERS-CoV (in 2012) and SARS-CoV-2 (in 2019) are viral species that can be pathological and fatal even in individuals immunologically competent.

SARS-CoV-2 is a cytopathic virus that induces the release of damage-associated molecular patterns (DAMPs) [Park W B, Kwon N J, Choi Si, Kang C K, Choe P G, Kim J Y, Yun J, Lee G W et al (2020) Virus isolation from the first patient with SARS-CoV-2 in Korea. J Korean Med Sci 35(7):e84]. DAMPs are endogenous molecules released from damaged cells that interact with molecules called pattern-recognition receptor (PRR) that induce in the neighboring epithelial cells, endothelial cells, and macrophages a state of high inflammation [Yilla M, Harcourt B H, Hickman O , McGrew M, Tamin A, Goldsmith C S, Bellini W J, Anderson U (2005) SARS coronavirus replication in human peripheral monocytes/macrophages. Virus Res 107(1):93-101].

During coronavirus disease 2019 (COVID-19) ˜5-10% of patients progress to acute respiratory distress syndrome (ARDS). The disease is caused by the severe acute respiratory syndrome coronavirus 2 (SARS-CoV-2). Although most patients infected with SARS-CoV-2 had a mild illness, about 5% of patients had severe lung injury or even multiorgan dysfunction, resulting in a 1.4% case fatality ratio. Severe COVID-19 is associated with a cytokine storm characterized by increased plasma concentrations of multiple cytokines including IL1β, IL2, IL6, IL7, IL8, IL10, IL17, IFNy, IFNy-inducible protein 10, monocyte chemoattractant protein 1 (MCP1), G-CSF, macrophage inflammatory protein 1α, and TNFα. Neutrophil infiltration can induce macrophages to secrete IL1β and that IL1β enhances. During a cytokine storm, a signaling loop between macrophages and neutrophils can lead to uncontrollable, progressive inflammation. It has been proposed that a neutrophil-IL1β loop is activated in severe COVID-19, leading to accelerated respiratory decompensation, the formation of microthrombi and aberrant immune responses.

COVID-19 is also associated with an immune suppression stage following the proinflammatory phase. It is characterized by sustained and substantial reduction of the peripheral lymphocyte counts, mainly CD4 T and CD8 T cells in COVID-19 patients and is associated with a high risk of developing secondary bacterial infection. Such a lymphopenia was also found in severe influenza and other respiratory viral infections. The degree of lymphopenia has been shown to correlate with the severity of COVID-19. The mechanism underlying lymphopenia remains unknown, but viral induced apoptosis or activation-induced apoptosis have been proposed. Previous studies have shown that SARS-like viral particles and SARS-CoV RNA were detected in T lymphocytes isolated from peripheral blood sample, spleen, lymph nodes, and lymphoid tissue of various organs, suggesting that SARS-CoV might be able to infect T cells directly.

Studies have revealed that 71.4% of non-survivors of COVID-19 present abnormal disseminated intravascular coagulation and showed abnormal coagulation results during later stages of the disease; particularly increased concentrations of D-dimer and other fibrin degradation products were significantly associated with poor prognosis. However, the concrete mechanisms for coagulopathy are not identified yet. Whether SARS-CoV-2 can directly attack vascular endothelial cells expressing high levels of ACE2 and then lead to abnormal coagulation and sepsis, still needs to be explored. Meanwhile, ACE2 is also an important regulator of blood pressure. High expression of ACE2 in the circulatory system after infection of SARS-CoV-2 might partially contribute to septic hypotension.

Severe cases of COVID-19 are characterized by a strong inflammatory process that may ultimately lead to organ failure and patient death. The NLR family pyrin domain containing 3 (NLRP3) is an important inflammasome sensor. In vitro, SARS-CoV-1 ORF 3a and protein E may induce the activation of NLRP3, and SARS-CoV-2 has been also found to activate NLRP3. The NLRP3 inflammasome is a molecular platform that promotes inflammation via cleavage and activation of key inflammatory molecules including active caspase-1 (Casp1p20), IL-1β and IL-18. Rodrigues et al. have shown that the NLRP3 inflammasome is activated in response to SARS-CoV-2 infection and it is active in COVID-19, influencing the clinical outcome of the disease (medRxiv; doi: https://doi.org/10.1101/2020.08.05.20168872 posted Aug. 6, 2020). Inflammasome-derived products such as Casp1p20 and IL-18 in the sera correlated with the markers of COVID-19 severity, including IL-6 and LDH. Moreover, higher levels of IL-18 and Casp1p20 are associated with disease severity and poor clinical outcome. this suggest that the inflammasome is key in the pathophysiology of the disease, indicating this platform as a marker of disease severity and a potential therapeutic target for COVID-19.

As both canonical and non-canonical inflammasomes exerts their effects hyperinflammatory effects, cytokine release amplification, and down-stream multiple organ pathogenesis disease through Caspase-1 and Caspase-4/5 activities, respectively, the core of this invention is the use of inhibitors of inflammatory caspases to prevent, reduce, or block these pathological sequences and in turn treat SARS, MERS, COVID-19, and other cytokine release syndrome (CRS), or cytokine storm syndrome (CSS)-related diseases (see FIG. 7).

SARS-CoV-2-induced inflammasome activation and pyroptosis in alveolar macrophages and recruited monocyte-derived macrophages could drastically aggravate symptoms of pneumonia, including acute respiratory distress syndrome and fever. (Jeremy K. Y. Yap, Miyu Moriyama and Akiko Iwasaki. Inflammasomes and Pyroptosis as Therapeutic Targets for COVID-19. J Immunol 2020, 205(2) 307-312).

It is predicted that pyroptosis in lung epithelial cells is likewise detrimental given the severe pneumonia experienced by COVID-19 patients. In contrast, pyroptosis in alveolar macrophage induces acute lung injury and exacerbates lung inflammation by promoting neutrophil infiltration into the lungs and augmented alveolar concentrations of cytokines IL-6, TNF-α, and IL-1β (He, X., et al., TLR4-upregulated IL-1β and IL-1RI promote alveolar macrophage pyroptosis and lung inflammation through an autocrine mechanism. Sci. Rep 2016, 6: 31663). The combination effects between leukocytosis and pyroptosis may be a major contributor to cytokine storms observed in COVID-19 patients. Another unsettling observation that is especially relevant to severe COVID-19 patients is that mechanical stretch of the lungs further amplifies lung inflammation via NLRP3 activation in alveolar macrophages and MAPK kinase 6—mediated high-mobility group box 1 (HMGB1) protein expression in alveolar epithelial cells. This reinforces the rationale of using a Caspase-1 inhibitor in COVID-19 patients subjected to oxygenation or mechanical ventilation.

The importance of NLRP3 in repressing SARS-CoV-2 virulence is emphasized in a study that demonstrated that significantly dampened NLRP3-mediated inflammation in bats conferred disease tolerance in these hosts, providing an ideal reservoir for a range of zoonotic viruses, including SARS-CoV, Middle East respiratory syndrome coronavirus, and likely SARS-CoV-2 (Ahn, M., et al. Dampened NLRP3-mediated inflammation in bats and implications for a special viral reservoir host. Nat. Microbiol. 2019, 4: 789-799). In fact, some viruses such as the influenza virus, measles virus, Sendai virus, and Nipah virus have evolved mechanisms to suppress the NLRP3 inflammasome (Zhao, C., W. Zhao. 2020. NLRP3 inflammasome-αkey player in antiviral responses. Front. Immunol. 11: 211).

The SARS-CoV genome encodes 3 ion channel proteins: E, open reading frame 3a (ORF3a), and ORF8a in which E and ORF3a are required for both replication and virulence (DeDiego M L, et al., Coronavirus virulence genes with main focus on SARS-CoV envelope gene. Virus Res. 2014 Dec. 19; 1940:124-37; Nieto-Torres J L, et al., Severe acute respiratory syndrome coronavirus envelope protein ion channel activity promotes virus fitness and pathogenesis. PLoS Pathog. 2014 May; 10(5):e1004077; Nieto-Torres J, et al. Severe acute respiratory syndrome coronavirus E protein transports calcium ions and activates the NLRP3 inflammasome. Virology. (2015) 485:330-9.; Lu W, et al., Severe acute respiratory syndrome-associated coronavirus 3α protein forms an ion channel and modulates virus release. Proc Natl Acad Sci USA. (2006) 103:12540-5.; Chen C C, et al., ORF8α of SARS-CoV forms an ion channel: experiments and molecular dynamics simulations. Biochim Biophys Acta. (2011) 1808:572-9.; Castaño-Rodriguez C, et al. . Role of severe acute respiratory syndrome coronavirus viroporins E, 3a, and 8a in replication and pathogenesis. mBio. (2018) 9:e02325-17).

In addition to the canonical NLRP3 activation pathway by PAMPs and DAMPs, the E, 3a, and 8b proteins of SARS-CoV function as NLRP3 agonists (Siu K L, et al., Severe acute respiratory syndrome coronavirus ORF3a protein activates the NLRP3 inflammasome by promoting TRAF3-dependent ubiquitination of ASC. FASEB J. (2019) 33:8865-77; Nieto-Torres J L, et al. . Severe acute respiratory syndrome coronavirus envelope protein ion channel activity promotes virus fitness and pathogenesis. PLoS Pathog. (2014) 10:e1004077.; Shi C S, et al., SARS-coronavirus open reading frame-8b triggers intracellular stress pathways and activates NLRP3 inflammasomes. Cell Death Discov. (2019) 5:101); many of these sequences are conserved in SARS-CoV-2 and likely play a role in inflammatory pathogenesis (Fung S Y, Yuen K S, Ye Z W, Chan C P, Jin D Y. A tug-of-war between severe acute respiratory syndrome coronavirus 2 and host antiviral defence: lessons from other pathogenic viruses. Emerg Microbes Infect. (2020) 9:558-70.). The SARS-CoV E, 3a, and 8b proteins are all reported to induce NLRP3 activation and IL-1β release in LPS-primed macrophage models (Chen I Y, Moriyama M, Chang M F, Ichinohe T. Severe acute respiratory syndrome coronavirus viroporin 3a activates the NLRP3 inflammasome. Front Microbiol. (2019) 10:50.). A wide variety of mechanisms have been proposed for this NLRP3 agonism including E-, 3a-, and 8b-induced viroporin activity, interferon antagonism, membrane-bound organelle stress, reactive oxygen species production, and direct binding to and regulation of inflammasome components such as caspase 1, NLRP3, and NE-KB (Yue Y, et al. . SARS-coronavirus open reading frame-3a drives multimodal necrotic cell death. Cell Death Dis. (2018) 9:904). There are multiple pathways by which SARS-CoV triggers NLRP3 activation which have yet to be characterized and are likely influenced by cell type and the extracellular microenvironment (Tan YJ, Lim SG, Hong W. Regulation of cell death during infection by the severe acute respiratory syndrome coronavirus and other coronaviruses. Cell Microbiol. (2007) 9:2552-61).

Hence we propose that both direct and indirect effect of viral proteins of SARS-CoV, MERS-CoV, and SARS-CoV-2 can activate inflammatory caspases, namely Caspase-1, Caspase-4 and/or Caspase-5 through inflammasome platforms and these effects may be key in the cytokine release syndrome or cytokine storm syndrome pathogenesis, of SARS, MERS and COVID-19, particularly in disease progression to severe condition.

Notably, the NLRP3-implicated ORFs 3a and 8 are the primary sites driving genetic diversification of SARS-CoV-2. ORF3a, specifically, is the only gene undergoing diversifying mutations that are predicted to exhibit altered phenotypes (Velazquez-Salinas L, Zarate S, Eberl S, Gladue D P, Novella I, Borca M V. Positive selection of ORF3a and ORF8 genes drives the evolution of SARS-CoV-2 during the 2020 COVID-19 pandemic. bioRxiv [preprint]. (2020). 10.1101/2020.04.10.035964). Ongoing mutations in ORF8 are particularly concerning, as a 29-nt deletion of the SARS-CoV genome is suspected to have increased the pathogenicity of the virus during the SARS-CoV epidemic by antagonizing interferon, increasing viral titers, and agonizing NLRP3. The uniquely low homology between SARS-CoV-2 and SARS-CoV ORFs 3a and 8 may play a role in the differences in virulence and pathogenesis between these two related viral infections (Velazquez-Salinas L, Zarate S, Eberl S, Gladue D P, Novella I, Borca M V. Positive selection of ORF3a and ORF8 genes drives the evolution of SARS-CoV-2 during the 2020 COVID-19 pandemic. bioRxiv [preprint]. (2020). 10.1101/2020.04.10.035964 [CrossRef] [Google Scholar]; Lau SK, et al. . Severe acute respiratory syndrome (SARS) Coronavirus ORF8 protein is acquired from SARS-related coronavirus from greater horseshoe bats through recombination. J Virol. (2015) 89:10532-47.; Pachetti M, et al. Emerging SARS-CoV-2 mutation hot spots include a novel RNA-dependent-RNA polymerase variant. J Transl Med. (2020) 18:179). Defining the inflammatory activities of these two proteins is therefore critical to predictive monitoring and modeling of novel SARS-CoV-2 strain emergence.

Genetic variations in host inflammasome pathways may also influence disease outcome. Mutations in the LRR domain of bat NLRP3 mediate an overall dampened NLRP3 response to agonists. In the context of coronavirus infections, MERS-CoV does not induce clinical disease in bats despite high viral titers; this appears to be mediated by NLRP3 (Ahn M, Anderson D E, Zhang Q, Tan C W, Lim B L, Luko K, et al. Dampened NLRP3-mediated inflammation in bats and implications for a special viral reservoir host. Nat Microbiol. (2019) 4:789-99). Interestingly, SARS-CoV ORF8b is reported to activate NLRP3 via direct binding to the LRR domain, suggesting a mechanism of coronavirus-induced NLRP3 activation and further indicating therapeutic potential for inflammatory Caspase inhibitors as immunomodulatory agents.

The known role of NLRP3 in hyperinflammatory acute respiratory distress syndrome (ARDS) and cytokine release syndrome (CRS), documented NLRP3 involvement in MERS-CoV and SARS-CoV severity, and apparent efficacy of anti-NLRP3 therapeutics in SARS-CoV and SARS-CoV-2 clinical trials and animal models strongly indicate that NLRP3 is a central mediator of severe COVID-19. (Tracey L. Freeman and Talia H. Swartz. Targeting the NLRP3 Inflammasome in Severe COVID-19. Front Immunol. 2020; 11: 1518).

Timing of therapy is critical as once individuals develop ARDS, the chances of improved outcomes with therapy are severely reduced. Targeted therapy for individuals with moderate disease before the development of respiratory failure will be critical. There is an urgent need to develop therapeutics that improve patient outcomes in severe COVID-19. Therefore, targeting this pathway through existing available therapeutic options would represent an important and viable approach to reducing SARS-CoV-2-induced inflammatory cytokine signaling and immediately improve patient outcomes.

There are numerous studies that implicate the NLRP3 inflammasome and IL-1β in mediating inflammation during lung injury and acute respiratory distress syndrome (ARDS) (Ganter M T, Roux J, Miyazawa B, Howard M, Frank J A, Su G, et al. Interleukin-1beta causes acute lung injury via alphavbeta5 and alphavbeta6 integrin-dependent mechanisms. Circ Res. (2008) 102:804-12; Patton L M, Saggart B S, Ahmed N K, Leff J A, Repine J E. Interleukin-1 beta-induced neutrophil recruitment and acute lung injury in hamsters. Inflammation. (1995) 19:23-9; Kolb M, Margetts P J, Anthony D C, Pitossi F, Gauldie J. Transient expression of IL-lbeta induces acute lung injury and chronic repair leading to pulmonary fibrosis. J Clin Invest. (2001) 107:1529-36). Bronchoalveolar fluid and plasma in patients with ARDS have elevated IL-1β levels compared to healthy controls (Patton L M, Saggart B S, Ahmed N K, Leff J A, Repine J E. Interleukin-1 beta-induced neutrophil recruitment and acute lung injury in hamsters. Inflammation. (1995) 19:23-9; Kolb M, Margetts P J, Anthony D C, Pitossi F, Gauldie J. Transient expression of IL-1beta induces acute lung injury and chronic repair leading to pulmonary fibrosis. J Clin Invest. (2001) 107:1529-36) and is associated with worse clinical outcomes. In other coronavirus infections including MERS-CoV and SARS-CoV, patients with ARDS had high levels of IL-1β, IL-6, and IL-8 (He L, Ding Y, Zhang Q, Che X, He Y, Shen H, et al. Expression of elevated levels of pro-inflammatory cytokines in SARS-CoV-infected ACE2+ cells in SARS patients: relation to the acute lung injury and pathogenesis of SARS. J Pathol. (2006) 210:288-97). In other respiratory viral infections such as influenza, high levels of IL-1β have been detected in bronchoalveolar fluid and plasma from patients with lung injury (91-94, 98-101). Furthermore, animal studies in which mice deficient in components of the inflammasome have reduced lung injury and enhanced survival with influenza infection (45, Schmitz N, Kurrer M, Bachmann M F, Kopf M. Interleukin-1 is responsible for acute lung immunopathology but increases survival of respiratory influenza virus infection. J Virol. (2005) 79:6441-8). In pharmacologic studies in which IL-1β or IL-1R was antagonized, influenza associated lung injury was reduced (Gasse P, Mary C, Guenon I, Noulin N, Charron S, Schnyder-Candrian S, et al. IL-1R1/MyD88 signaling and the inflammasome are essential in pulmonary inflammation and fibrosis in mice. J Clin Invest. (2007) 117:3786-99.; Kim K S, Jung H, Shin I K, Choi B R, Kim D H. Induction of interleukin-1 beta (IL-1(3) is a critical component of lung inflammation during influenza A (H1N1) virus infection. J Med Virol. (2015) 87:1104-12). However, targeting Caspase-1 allow to inhibit the deleterious cascade upstream of cytokine release and thus to avoid the caveat of inhibiting only one cytokine among many.Taken together, IL-1β appears to play a key role in acute lung injury with respiratory viral infections and pharmacologic targeting of this pathway represents an important area of intervention.

Injury of type II alveolar epithelial cells expressing ACE2 leads to NLRP3 inflammasome activation (Zhao C, Zhao W. NLRP3 inflammasome-a key player in antiviral responses. Front Immunol. (2020) 11:211) (Chen IY, Moriyama M, Chang MF, Ichinohe T. Severe acute respiratory syndrome coronavirus viroporin 3a activates the NLRP3 inflammasome. Front Microbiol. (2019) 10:50, 105). The acute immune response to SARS-CoV-2 infection is largely driven by inflammatory alveolar and monocyte-derived macrophages that are activated by PAMPs and DAMPs released by infected, apoptotic pneumocytes (Tisoncik J R, Korth M J, Simmons C P, Farrar J, Martin T R, Katze M G. Into the eye of the cytokine storm. Microbiol Mol Biol Rev. (2012) 76:16-32). TNF-α and IL-1β secreted by alveolar macrophages initiate the acute proinflammatory cascade immediately following infection. The secretion of these cytokines induces cell death and damage, PAMP/DAMP production, immune cell recruitment, and widespread NLRP3 activation, establishing a proinflammatory positive feedback cascade. More recently, Blanco-Melo et al. demonstrated that SARS-CoV-2 infection of primary human bronchial epithelial cells resulted in expression of multiple cytokines and chemokines including TNF-α, IL-6, and IL-1β (Blanco-Melo D, Nilsson-Payant B E, Liu W C, Uhl S, Hoagland D, Moller R, et al. Imbalanced host response to SARS-CoV-2 drives development of COVID-19. Cell. (2020) 181:1036-45.e9).

This localized inflammatory cell death extends to the vasculature, inducing the leakage, edema, and pneumonia characteristic of COVID-19. It is important to note that the onset of this pathological immune response is characterized not by systemic inflammation, but by a hyperinflammatory microenvironment localized to the site of tissue injury. As the inflammatory cascade progresses, IL-1β, and TNF-α induce the secretion of additional NLRP3 cytokines such as IL-6 which can subsequently be observed in the peripheral blood due to the loss of vascular integrity. The kinetics of the inflammatory response are essential to effective clinical practice—circulating biomarkers such as IL-6 may prove useful to predicting outcomes and informing immunomodulatory treatment decisions.

The rapid decline of COVID-19 patients coincides with an abrupt shift from the NLRP3 cytokine storm to a compensatory immunosuppressive state (Mehta P, McAuley D F, Brown M, Sanchez E, Tattersall R S, Manson J J, et al. COVID-19: consider cytokine storm syndromes and immunosuppression. Lancet. (2020) 395:1033-1034.). This repair and recovery-oriented phase is characterized by production of IL-10, polarization of macrophages to the anti-inflammatory M2 state, suppression of NLRP3, and recruitment of fibroblasts and platelets. The accumulation of fibroblasts and M2 macrophages in the lung initiates the deposition of collagen and construction of the extracellular matrices that characterize ARDS fibrosis. M2 macrophages and other markers of this pro-fibrotic, anti-inflammatory environment have been detected in the bronchioalveolar fluid of severe COVID-19 patients. Importantly, an essential viroporin encoded by ORF3a, required for release of SARS-CoV-2 from infected cells is also able to prime and activate the NLRP3 inflammasome, the machinery responsible for much of the inflammatory pathology in severely ill patients. ORF3a triggers IL-1β expression via NFκB, thus priming the inflammasome while also activating it via ASC-dependent and independent modes. ORF3a-mediated inflammasome activation requires efflux of potassium ions and oligomerization between NEK7 and NLRP3 (Xu, H., Chitre, S., Akinyem I., Loeb, J. et al. (2020). SARS-CoV-2 viroporin triggers the NLRP3 inflammatory pathway. bioRxiv (https://www.biorxiv.org/content/10.1101/2020.10.27.357731v1).

Unique to SARS-CoV and SARS-CoV-2 is the downmodulation of the ACE2 receptor. SARS-CoV entry has been reported to be dependent on TNF-α converting enzyme and coupled to the release of TNF-α from the cell membrane. TNF-a, specifically, has been shown to act as an alternative toll-like receptor (TLR) agonist that may increase the sensitivity and longevity of NLRP3 activation. Downregulation of ACE2 is associated with both SARS-CoV and SARS-CoV-2 disease severity; this contrasts with a minimally symptomatic coronavirus strain, HCoV-NL63, that utilizes but does not cleave or downmodulate the ACE2 receptor. The overproduction of TNF-α in COVID-19 may preferentially activate the NLRP3 inflammasome relative to other immunological pathways.

Today, there is no antiviral drug or no other specific treatment to prevent and/or treat coronavirus infection and its immuno-inflammatory consequences. Today, there is no specific treatment to prevent or treat SARS, MERS or COVID-19, nor cytokine release syndrome or cytokine storm syndrome.

Consequently, it appears necessary to have new compositions capable of inhibiting inflammatory caspases for ameliorating, preventing or treating coronavirus infection, MERS, SARS, COVID-19, cytokine release syndrome (CRS), or cytokine storm syndrome (CSS), while reducing the risk of side effects, when administered to the patient.

In addition, a clinical use of Belnacasan and other inhibitors of inflammatory Caspases has been underestimated because when Belnacasan and derivative have been discovered, the role of human caspase-4, human caspase-5 and the mouse orthologue caspase-11, in the non-canonic inflammasome pathway was not yet understood. And consequently, sepsis was considered not fully dependent on inflammatory Caspases. It is now known that human Caspase-4 (or alternatively human Caspase-5) can be direct receptors of intracellular LPS and critical for various hyperinflammatory conditions particularly but not limited to intracellular pathogens. The use of inhibitors that target specifically or at least preferentially the 3 major human inflammatory Caspases (Caspase-1, Caspase-4, Caspase-5) such as Belnacasan has been underestimated for the treatment of acute inflammatory conditions where both canonic (Caspase-1 and its activation platform) and non-canonic (Caspase-4/-5 and its activation platform) inflammasomes are critically implicated, for instance Coronaviruses-induced acute inflammatory syndrome, intracellular bacteria-induced septic shock, or brain sepsis.

### SUMMARY OF THE INVENTION

The present invention relates to methods to inhibit the inflammatory caspases for ameliorating, preventing, or treating diseases associated with excessive inflammatory responses to pathogenic infection or danger signalization including coronaviruses infection and inflammatory consequences of coronaviruses infection in humans, SARS, MERS, COVID-19. The identification of a central role of the inflammatory Caspases in the pathogenic activity of the canonic and non-canonic inflammasomes provide a general method to treat acute diseases including cytokine release syndrome (CRS), or cytokine storm syndrome (CSS), multi-organ failure, and pathogen induced acute respiratory distress syndromes. Compounds to be repurposed and new pharmaceutical composition are claimed.

Advantageously, the present invention relates to a method for ameliorating, treating, or preventing coronavirus infection, coronavirus disease 2019 (COVID-19), Multisystem Inflammatory Syndrome in Children (MIS-C) associated with coronavirus disease 2019 (COVID-19), Severe Acute Respiratory Syndrome (SARS), Middle East Respiratory Syndrome (MERS), pathogen-related respiratory distress syndrome, pathogen-related multi-organ failure, cytokine release syndrome, cytokine storm syndrome, or diseases associated with excessive activation of the canonic and non-canonic inflammasomes, comprising administering to a patient in need of such treatment an effective amount of an inhibitor of inflammatory caspases characterized in that it selectively or preferentially inhibits at least one or all the so-called inflammatory Caspases including human Caspase-1, human Caspase-4, and human Caspase-5.

Advantageously, said inhibitor of inflammatory caspases is selected among a group comprising:

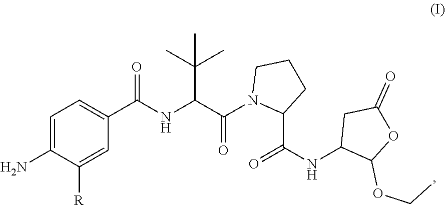

and each stereoisomer thereof, including:

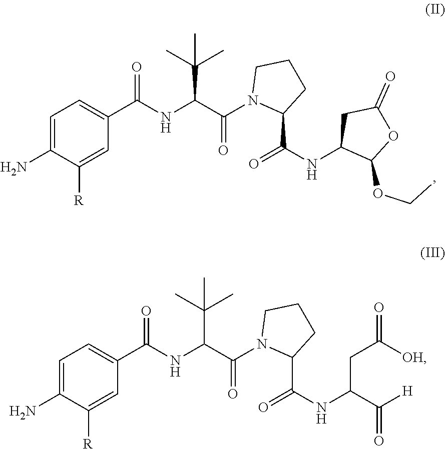

and each stereoisomer thereof, including:

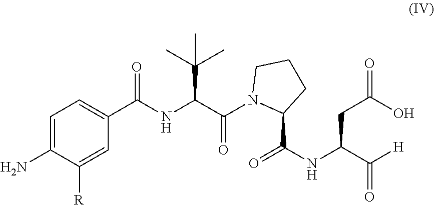

wherein R is H, OH, CH₃, Cl, or another halogen, or a pharmaceutical salt,

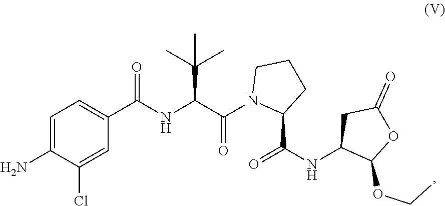

(also known as Belnacasan or (2S)-1-[(2S)-2-[(4-amino-3-chlorobenzoyl)amino]-3,3-diethylbutanoyl]-N-[(2R,3S)-2-ethoxy-5-oxooxolan-3-yl]pyrrolidine-2-carboxamide),
or a pharmaceutical salt thereof as defined above,

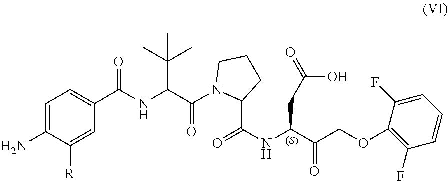

wherein R is H, OH, CH₃ or a halogen, or a pharmaceutical salt,

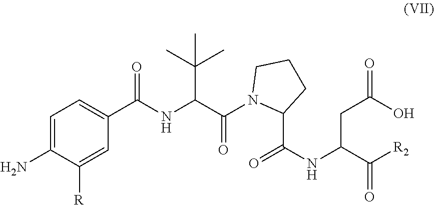

wherein R is H, OH, CH₃ or a halogen, or a pharmaceutical salt, and wherein R2 is

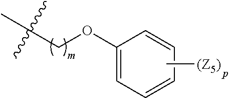

in which m is 0, 1 or 2
Z is a halogen,
p is 1, 2, 3, 4, or 5;
or a pharmaceutically acceptable salt thereof; and

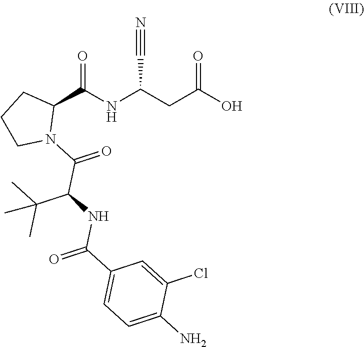 [SMILES: CC(C)(C)[C@H](NC(=O)c1ccc(N)c(Cl)c1)C(=O)N1CCC[C@H]1C(=O)N[C@H](C#N)CC(=O)O]

(also known as (S)-3-((S)-1-((S)-2-(4-amino-3-chlorobenzamido)-3,3-dimethylbutanoyl)pyrrolidine-2-carboxamido)-3-cyanopropanoic acid) or a pharmaceutically acceptable salt thereof, or a pharmaceutical composition containing either entity.

In one embodiment, said inhibitor of inflammatory caspases, salt or composition is administered to the patient by oral, parenteral, intravenous, intramuscular, intranasal, sublingual, intratracheal, inhalation, ocular, vaginal, and rectal route. Advantageously, the pharmaceutical composition is in a suitable form for its administration by oral, intravenous, parenteral, intramuscular, intranasal, sublingual, intratracheal, inhalation, ocular, vaginal, and rectal route.

In one embodiment, the method comprises administering one or more than one intravenous doses of a therapeutically effective dose of said inhibitor of inflammatory caspases.

In one embodiment, the method further comprises at least one pharmaceutically acceptable carrier.

In one embodiment, said inhibitor of inflammatory caspases is administered to the patient at a dose of 300 mg to 2,400 mg per administration, advantageously at a dose of 600 mg to 1,800 mg per administration, advantageously at a dose of 900 mg per administration.

In one embodiment, said inhibitor of inflammatory caspases is administered to the patient at a dose of 2 to 200 mg/kg of body weight/day, advantageously at a dose of 6 to 100 mg/kg of body weight/day, advantageously at a dose of 25 to 75 mg/kg of body weight/day.

In one embodiment, the method further comprises administering a second active ingredient selected among N-Acetyl-cystein, Fibrin-derived peptide B1315-42, Vitamin D, Molnupiravir, or SARS-CoV-2 protease inhibitors including PF-07321332 and PF-07304814.

In one embodiment, the method further comprises administering an antibiotic or several antibiotics among ceftriaxone, spiramycin, amoxicillin, amoxicillin/clavulanic acid, gentamicin, netilmicin, piperacilin/tazobactam, amikacin, cefuroxime, penicillin, azithromycin, clarithromycin, erythromycin, doxycycline, cefotaxime, ampicillin, Ertapenem, cefepime, imipenem, meropenem, metronidazole, fluconazole, ciprofloxacin, levofloxacin, vancomycin, linezolid, moxifloxacin and gemifloxacin.

In one embodiment, the patient is a patient infected by a coronavirus or has been admitted to hospital following a PCR detection of a pathogenic coronavirus. Advantageously, the patient is infected by the SARS-CoV-2 or has been admitted to hospital following a PCR detection of SARS-CoV-2. Advantageously, the patient is admitted to intensive care unit with elevated markers inflammation, low oxygen levels, or acute respiratory distress.

The present invention also relates to a pharmaceutical composition comprising a compound that inhibits selectively or preferentially at least one or all the so-called inflammatory Caspases including human Caspase-1, human Caspase-4, and human Caspase-5, for ameliorating, treating, or preventing coronavirus infection, coronavirus disease 2019 (COVID-19), multisystem inflammatory syndrome in children (MIS-C) associated with coronavirus disease 2019 (COVID-19), Severe Acute Respiratory Syndrome (SARS), Middle East Respiratory Syndrome (MERS), pathogen-related respiratory distress syndrome, pathogen-related multi-organ failure, cytokine release syndrome, cytokine storm syndrome, or diseases associated with excessive activation of the canonic and non-canonic inflammasomes.

Advantageously, the present invention relates to a pharmaceutical composition comprising an inhibitor of inflammatory caspases that inhibits selectively or preferentially at least one or all the so-called inflammatory Caspases including human Caspase-1, human Caspase-4, and human Caspase-5, for ameliorating, treating, or preventing coronavirus infection, coronavirus disease 2019 (COVID-19), multisystem inflammatory syndrome in children (MIS-C) associated with coronavirus disease 2019 (COVID-19), Severe Acute Respiratory Syndrome (SARS), Middle East Respiratory Syndrome (MERS), pathogen-related respiratory distress syndrome, pathogen-related multi-organ failure, cytokine release syndrome, cytokine storm syndrome, or diseases associated with excessive activation of the canonic and non-canonic inflammasomes.

Advantageously, said inhibitor of inflammatory caspases is selected among:

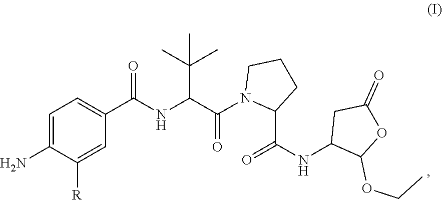

and each stereoisomer thereof, including:

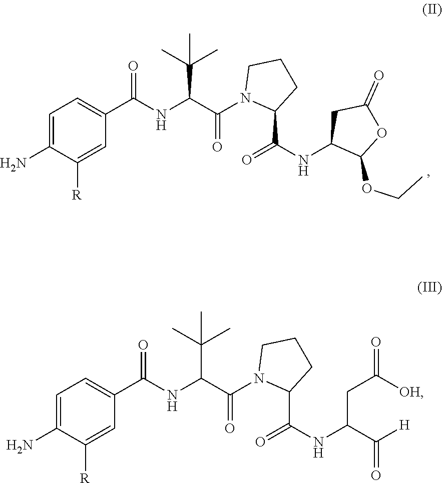

and each stereoisomer thereof, including:

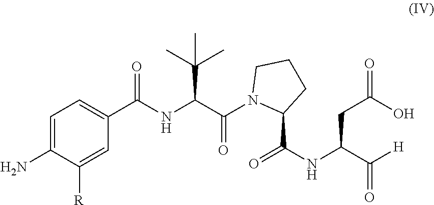

wherein R is H, OH, CH₃, CI, or another halogen, or a pharmaceutical salt,

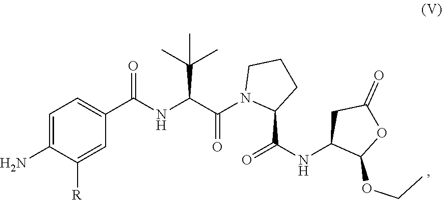

(also known as Belnacasan or (2S)-1-[2S-2-[(4-amino-3-chlorobenzoyl)a no]-3,3-diethylbutanoyl]-N-[(2R,3S)-2-ethoxy-5-oxooxolan-3-yl]pyrrolidine-2-carboxamide),
or a pharmaceutical salt thereof as defined above.

In one embodiment, the inhibitor of inflammatory caspases selected among the group comprising:

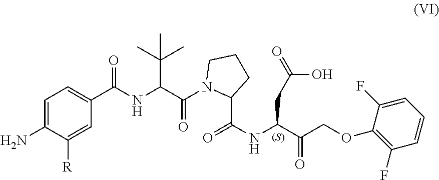

wherein R is H, OH, CH₃ or a halogen, or a pharmaceutical salt;

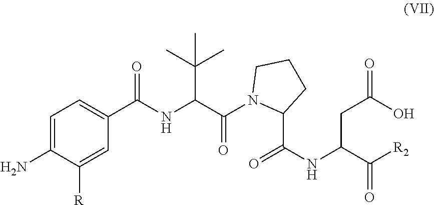

wherein R is H, OH, CH₃ or a halogen, or a pharmaceutical salt, and wherein R2 is

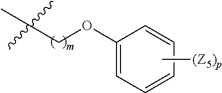

in which m is 0, 1 or 2
Z is a halogen,
p is 1, 2, 3, 4, or 5; and

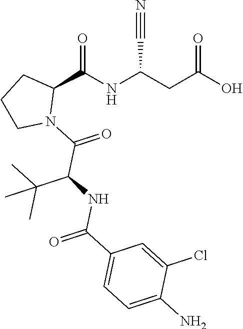 [SMILES: CC(C)(C)[C@H](NC(=O)c1ccc(N)c(Cl)c1)C(=O)N1CCC[C@H]1C(=O)N[C@H](C#N)CC(=O)O]

(also named (S)-3-((S)-1-((S)-2-(4-amino-3-chlorobenzamido)-3,3-dimethylbutanoyl)pyrrolidine-2-carboxamido)-3-cyanopropanoic acid) or a pharmaceutical salt thereof.

In one embodiment, the pharmaceutical composition is administered by oral, intravenous, parenteral, intramuscular, intranasal, sublingual, intratracheal, inhalation, ocular, vaginal, and rectal route. Advantageously, the pharmaceutical composition is in a suitable form for its administration by oral, intravenous, parenteral, intramuscular, intranasal, sublingual, intratracheal, inhalation, ocular, vaginal, and rectal route.

In one embodiment, the pharmaceutical composition is administered to the patient at a dose of 2 to 200 mg/kg of body weight/day, advantageously at a dose of 6 to 100 mg/kg of body weight/day, advantageously at a dose of 25 to 75 mg/kg of body weight/day.

In one embodiment, the pharmaceutical composition further comprises a second active ingredient. Advantageously, the second active ingredient is selected among N-Acetyl-cysteine, Fibrin-derived peptide Bf315-42, Vitamin D, and Molnupiravir. Advantageously, the second active ingredient includes one or several antibiotics among: ceftriaxone, spiramycin, amoxicillin, amoxicillin/clavulanic acid, gentamicin, netilmicin, piperacilin/tazobactam, amikacin, cefuroxime, penicillin, azithromycin, larithromycin, erythromycin, doxycycline, cefotaxime, ampicillin, Ertapenem, cefepime, imipenem, meropenem, metronidazole, fluconazole, ciprofloxacin, levofloxacin, vancomycin, linezolid, moxifloxacin and gemifloxacin.

The present invention also relates to a method for reduction of Caspase-1 activation, Caspase-4 activation, Caspase-5 activation, IL-1β maturation and release, IL-18 maturation, Pyroptosis, and release or other biomarkers in a patient having coronavirus infection, coronavirus disease 2019 (COVID-19), multisystem inflammatory syndrome in children (MIS-C) associated with coronavirus disease 2019 (COVID-19), Severe Acute Respiratory Syndrome (SARS), Middle East Respiratory Syndrome (MERS), pathogen-related respiratory distress syndrome, pathogen-related multi-organ failure, cytokine release syndrome, cytokine storm syndrome, or diseases associated with excessive activation of the canonic and non-canonic inflammasomes, comprising the step of administering to a patient said pharmaceutical composition.

### DESCRIPTION OF THE FIGURES

FIG. 1: Inflammasome activation influences the clinical outcome of COVID-19. (A). Correlation matrix of Casp1p20 and IL-18 levels in the serum of COVID-19 patients at the hospitalization day with patient characteristics and clinical parameters. (B-J) Correlations of Casp1p20 with IL-18 (B), Casp1p20 with IL-6 (C), Casp1p20 with lactate dehydrogenase (LDH) (D), Casp1p20 with C-reactive protein (CRP), IL-18 with IL-6 (F) and IL-18 with CRP (G). (H,I) Levels of Casp1p20 (H) and IL-18 (I) in patients that required (MV+, blue box) or not (MV-, red box) mechanical ventilation. (J,K) Levels of Casp1p20 (J) and IL-18 (K) in patients with Mild/Moderate (yellow box) or Severe COVID-19 (pink box). (L,M) Levels of Casp1p20 (L) and IL-18 (M) in survivors (green box) or non-survivors (purple box). The levels of Casp1p20 and IL-18 were measured by ELISA and are shown as Log10-transformed concentrations in pg/mL. *P<0.05, **P <0.01 and ***P <0.001 as determined by Student's t-test. Each dot 584 represents value to form a single individual. Box shows average +/−SD of the values. (N, O) Derived predictions from the best-fit models retained in Casp1p20 (N) and IL-18 (O) longitudinal analyses; IL-18 Model (O) comprises variation in the intercept among patients' groups: Death (Black), Critical/Recovery (Grey) and Mild/Recovery (Grey).

FIG. 2: Cytokine production in COVID-19 patients. Cytokine concentration in the serum control individuals (CT, n=45) and COVID-19 patients (COVID-19 P, n=92; all tested positive using RT-PCR). IL-10 (A), IL-4 (B), IFN-g (C), TNF-α (D) and IL-17A (E) were measured by CBA. Data are shown as Log10-transformed concentrations in pg/mL. **P <0.01 and ***P<0.001 as determined by Student's t test. Each dot represents the value form a single individual. Box show average+/−SD of the values.

FIG. 3: Quantification of the presence of the inflammasome, expressed as ASC specks per high-power fields, showing a significantly higher number of inflammasomes in COVID-19 patients versus controls. Data expressed as mean and standard deviation. P value generated using 2-tails T test for unpaired samples.

FIG. 4: SARS-Coronavirus Open Reading Frame-8b triggers intracellular stress pathways and activates NLRP3 inflammasomes. (a) Immunoblots for the indicated proteins of PMA differentiated THP-1 cell supernatant and lysates after transient transfection of expression vectors for GFP, 8b-GFP, GFP-8b, or GFP-8b V77K. (b) Immunoblots for the indicated proteins of PMA differentiated THP-1 cell supernatant and lysates after transient transfection of Flag or 8b-Flag. (c) Immunoblots for the indicated proteins of HEK 293T cell supernatants and lysates expressing the requisite inflammasome components and either GFP-8b or 8b-Flag with their appropriate control vectors. (d) Confocal microscopy images of HeLa cells expression NLRP3-DsRed only or NLRP3-DsRed and GFP-8b. Nuclei were counterstained with DAPI and cells were fixed prior to imaging. Individual and merged images are shown, and the scale bar is 5 μm. (e) Confocal microscopy images of HEK 293T cells expressing NLRP3-DsRed and GFP-8b. Left panels show co-transfected cell and a cell expressing NLRP3-DsRed alone. The right panels show a cell expressing both NLRP3-DsRed and GFP-8b. Individual and merged images are shown, and the scale bar is 5 μm. (f) Confocal microscopy images of HeLa cells expressing NLRP3-DsRed and GFP-8b and immunostained for TGN38. Individual and merged images are shown, and the scale bar is 3 μm. Images from a single confocal slice. Arrows point areas of overlapping signal. (g) Confocal microscopy images of THP-1 cells LPS activated and transfected with either GFP or GFP-8b. The following day the cells were immunostained for NLRP3 (upper panels) or ASC (lower panels). Individual and merged images are shown, and the scale bar is 5 μm. Images and blots are representative data from experiments repeated a minimum of 3 times. Of note additional data indicates that data indicates that ORF8b directly targets NLRP3 by binding to its LRR domain.

FIG. 5: Evaluation of cytotoxicity and IL-1-cytokine release for increasing doses of nigericin after 30, 60 and 120 minutes of incubation. (A) Effect of nigericin on cell death at 5 μM, (B) 10 μM and (C) 20 μM for different incubation times. (D) Nigericin causes the release of IL-1β (pg/mL) in the medium after cell differentiation with phorbol esters (PMA; 50 nM) for 24 h and stimulation for 6 hours with 1 μg/mL of LPS. In this model, after 120 minutes of Nigericin treatment, VX-765 (yellow) at 25 μM makes it possible to reduce the release of the cytokine into the medium to 90%.

FIG. 6: VX-765 inhibits the inflammasome-mediated release of IL1beta. Belnacasan (VX-765) prevents the release of IL-lbeta induced by the treatment with nigericin. Cell differentiation was induced with phorbol esters (PMA; 50 nM) for 24h and stimulation for 6 h with 1 μg/mL of LPS CA) Nigericin at 5 μM for 60 and 120 minutes. (n=3).

FIG. 7: Representative schema of inhibition role of VX-765.

### DESCRIPTION OF THE INVENTION & DEFINITIONS

The present invention provides pharmaceutical compositions that are particularly effective for ameliorating, preventing or treating SARS, MERS, COVID-19, cytokine release syndrome, cytokine storm syndrome, inflammasome-related multi-organ failure, and pathogen-induced acute respiratory distress syndromes.

Advantageously, the present invention is directed to a pharmaceutical composition comprising an inhibitor of inflammatory caspases selected among the group comprising:

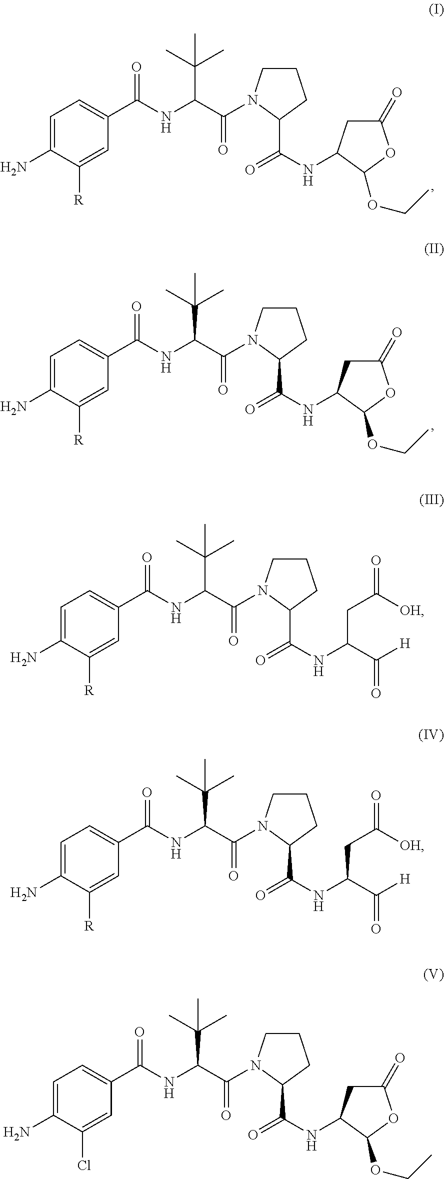

(also named (2S)-1-[(25)-2-[(4-amino-3-chlorobenzoyl)amino]-3,3-diethylbutanoyl]-N-[(2R,35)-2-ethoxy-5-oxooxolan-3-yl]pyrrolidine-2-carboxamide)

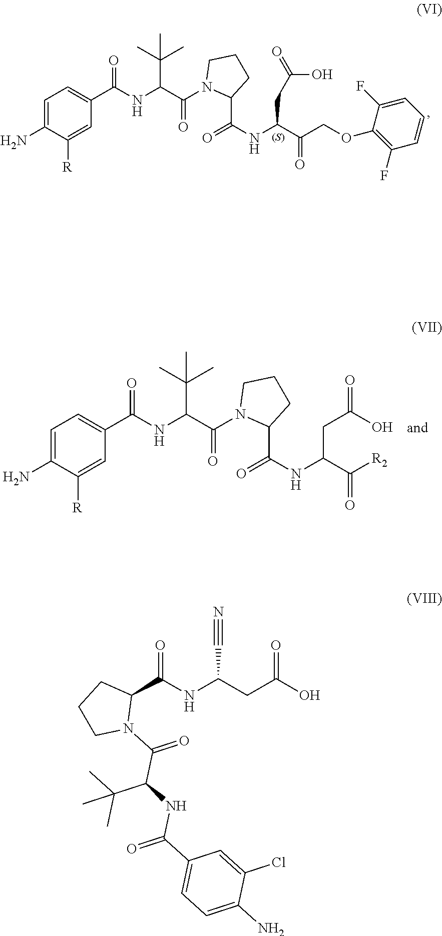

(also named (S)-3-((S)-1-((S)-2-(4-amino-3-chlorobenzamido)-3,3-dimethylbutanoyl)pyrrolidine-2-carboxamido)-3-cyanopropanoic acid),
wherein R is H OH, CH₃ or an halogen, and wherein R2 is

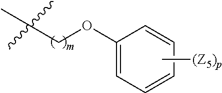

in which m is 0, 1 or 2
Z is a halogen,
p is 1, 2, 3, 4, or 5;
or a pharmaceutically acceptable salt thereof,
to prevent, treat or ameliorate coronavirus infection, coronavirus disease 2019 (COVID-19), multisystem inflammatory syndrome in children (MIS-C) associated with coronavirus disease 2019 (COVID-19), Severe Acute Respiratory Syndrome (SARS), Middle East Respiratory Syndrome (MERS), pathogen-related respiratory distress syndrome, pathogen-related multi-organ failure, cytokine release syndrome, cytokine storm syndrome, or diseases associated with excessive activation of the canonic and non-canonic inflammasomes, comprising administering to a patient in need of such treatment.

As used herein, the term “inhibit” refers to a decrease in an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10%, advantageously a 20%, advantageously a 30%, advantageously a 40%, advantageously a 50%, advantageously a 60%, advantageously a 70%, advantageously a 80%, advantageously a 90%, advantageously a 100%, or any amount of reduction in between as compared to native or control levels. Advantageously, the inhibitor of inflammatory caspases can reduce the activity of caspase-1 or caspase-4 or caspase-5 by 10%, advantageously by 20%, advantageously by 30%, advantageously by 40%, advantageously by 50%, advantageously by 60%, advantageously by 70%, advantageously by 80%, advantageously by 90%, advantageously by 100%. By targeting Caspase-1, the inhibitor of the invention allows to inhibit the deleterious cascade upstream of cytokine release and thus to avoid the caveat of inhibiting only one cytokine among many.

As used herein, the term “inflammatory caspases” refers to caspase-1, caspase-4, caspase-5 or a mixture thereof. Advantageously, the inhibitor of inflammatory caspases of the invention is able to inhibit caspase-1. Advantageously, the inhibitor of inflammatory caspases of the invention is able to inhibit caspase-4. Advantageously, the inhibitor of inflammatory caspases of the invention is able to inhibit caspase-5. Advantageously, the inhibitor of inflammatory caspases of the invention is able to inhibit caspase-1 and caspase-4. Advantageously, the inhibitor of inflammatory caspases of the invention is able to inhibit caspase-1 and caspase-5. Advantageously, the inhibitor of inflammatory caspases of the invention is able to inhibit caspase-5 and caspase-4. Advantageously, the inhibitor of inflammatory caspases of the invention is able to inhibit caspase-1, caspase-4 and caspase-5.

Surprisingly, the inventors have shown that the inhibitor of inflammatory caspases, and in particular inhibitors of Caspase-1, Caspase-4 and/or Caspase-5 may be used to inhibit pathogenic progression from mild/moderate to severe cases of coronavirus infection.

Surprisingly, the inventors have shown that the inhibitor of inflammatory caspases, and in particular inhibitors of Caspase-1, Caspase-4 and/or Caspase-5 is able to reduce several inflammasomes, and in particular NLRP3 inflammasome and inflammasome associated with high level of Casp1p20 and IL-18.

In a particular embodiment of the invention, the pharmaceutical composition of the invention comprises the inhibitor of inflammatory caspases of formula (I)

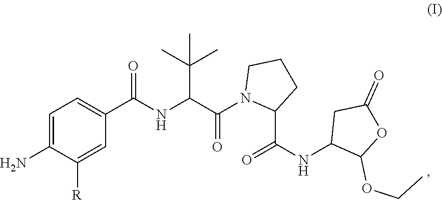

wherein R is H, CH₃, CI, or another halogen, or a pharmaceutical salt thereof, in effective amount for ameliorating, preventing or treating coronavirus infection, coronavirus disease 2019 (COVID-19), Multisystem Inflammatory Syndrome in Children (MIS-C) associated with coronavirus disease 2019 (COVID-19), Severe Acute Respiratory Syndrome (SARS), Middle East Respiratory Syndrome (MERS), pathogen-related respiratory distress syndrome, pathogen-related multi-organ failure, cytokine release syndrome, cytokine storm syndrome, or diseases associated with excessive activation of the canonic and non-canonic inflammasomes.

In another particular embodiment of the invention, the pharmaceutical composition of the invention comprises the inhibitor of inflammatory caspases of formula (II)

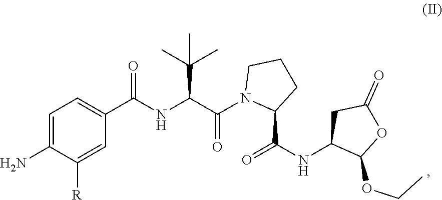

wherein R is H, CH₃, CI or another halogen, or a pharmaceutical salt thereof, in effective amount for ameliorating, preventing or treating coronavirus infection, coronavirus disease 2019 (COVID-19), Multisystem Inflammatory Syndrome in Children (MIS-C) associated with coronavirus disease 2019 (COVID-19), Severe Acute Respiratory Syndrome (SARS), Middle East Respiratory Syndrome (MERS), pathogen-related respiratory distress syndrome, pathogen-related multi-organ failure, cytokine release syndrome, cytokine storm syndrome, or diseases associated with excessive activation of the canonic and non-canonic inflammasomes.

In another particular embodiment of the invention, the pharmaceutical composition of the invention comprises the inhibitor of inflammatory caspases of formula (III)

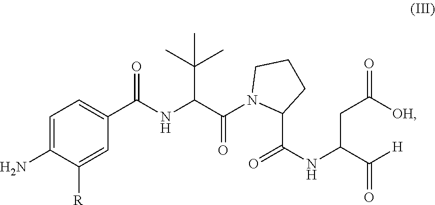

wherein R is H, CH₃ or an halogen being preferably but not limited to CI or F, or a pharmaceutical salt thereof, in effective amount for ameliorating, preventing or treating coronavirus infection, coronavirus disease 2019 (COVID-19), Multisystem Inflammatory Syndrome in Children (MIS-C) associated with coronavirus disease 2019 (COVID-19), Severe Acute Respiratory Syndrome (SARS), Middle East Respiratory Syndrome (MERS), pathogen-related respiratory distress syndrome, pathogen-related multi-organ failure, cytokine release syndrome, cytokine storm syndrome, or diseases associated with excessive activation of the canonic and non-canonic inflammasomes.

In another particular embodiment of the invention, the pharmaceutical composition of the invention comprises the inhibitor of inflammatory caspases of formula (IV)

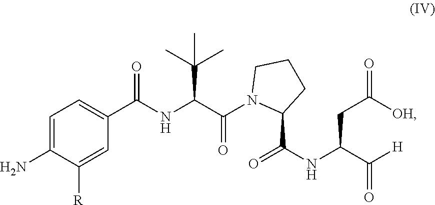

wherein R is H, CH₃, CI or another halogen, or a pharmaceutical salt thereof, in effective amount for ameliorating, preventing, or treating coronavirus infection, coronavirus disease 2019 (COVID-19), Multisystem Inflammatory Syndrome in Children (MIS-C) associated with coronavirus disease 2019 (COVID-19), Severe Acute Respiratory Syndrome (SARS), Middle East Respiratory Syndrome (MERS), pathogen-related respiratory distress syndrome, pathogen-related multi-organ failure, cytokine release syndrome, cytokine storm syndrome, or diseases associated with excessive activation of the canonic and non-canonic inflammasomes.

In a particular embodiment of the invention, the pharmaceutical composition of the invention comprises the inhibitor of inflammatory caspases of formula (V)

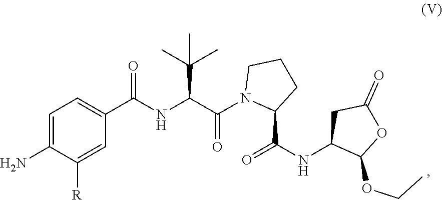

wherein R is H, CH₃ or an halogen, or a pharmaceutical salt thereof, in effective amount for ameliorating, preventing or treating coronavirus infection, coronavirus disease 2019 (COVID-19), Multisystem Inflammatory Syndrome in Children (MIS-C) associated with coronavirus disease 2019 (COVID-19), Severe Acute Respiratory Syndrome (SARS), Middle East Respiratory Syndrome (MERS), pathogen-related respiratory distress syndrome, pathogen-related multi-organ failure, cytokine release syndrome, cytokine storm syndrome, or diseases associated with excessive activation of the canonic and non-canonic inflammasomes.

In another particular embodiment of the invention, the pharmaceutical composition of the invention comprises the inhibitor of inflammatory caspases of formula (VI):

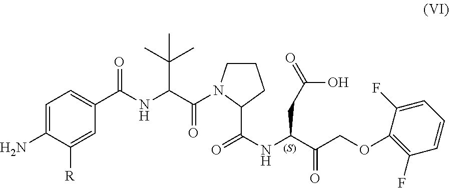

wherein R is H, OH, CH₃ or a halogen, or a pharmaceutical salt,or a pharmaceutical salt thereof, in effective amount for ameliorating, preventing or treating coronavirus infection, coronavirus disease 2019 (COVID-19), Multisystem Inflammatory Syndrome in Children (MIS-C) associated with coronavirus disease 2019 (COVID-19), Severe Acute Respiratory Syndrome (SARS), Middle East Respiratory Syndrome (MERS), pathogen-related respiratory distress syndrome, pathogen-related multi-organ failure, cytokine release syndrome, cytokine storm syndrome, or diseases associated with excessive activation of the canonic and non-canonic inflammasomes.

In another particular embodiment of the invention, the pharmaceutical composition of the invention comprises the inhibitor of inflammatory caspases of formula (VII)

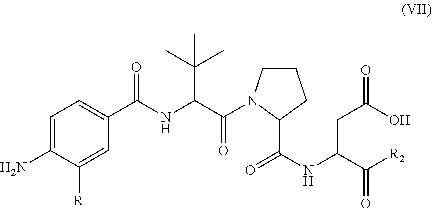

wherein R is H, OH, CH₃ or a halogen, or a pharmaceutical salt, and wherein R2 is

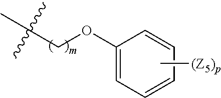

In which m is 0, 1 or 2

Z is a halogen,
p is 1, 2, 3, 4, or 5,
or a pharmaceuticalsalt thereof, in effective amount for ameliorating, preventing or treating coronavirus infection, coronavirus disease 2019 (COVID-19), Multisystem Inflammatory Syndrome in Children (MIS-C) associated with coronavirus disease 2019 (COVID-19), Severe Acute Respiratory Syndrome (SARS), Middle East Respiratory Syndrome (MERS), pathogen-related respiratory distress syndrome, pathogen-related multi-organ failure, cytokine release syndrome, cytokine storm syndrome, or diseases associated with excessive activation of the canonic and non-canonic inflammasomes.

In a particular embodiment of the invention, the pharmaceutical composition of the invention comprises the inhibitor of inflammatory caspases of formula (VIII)

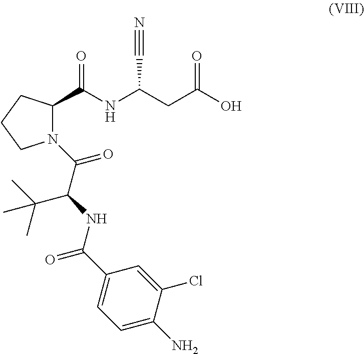 [SMILES: CC(C)(C)[C@H](NC(=O)c1ccc(N)c(Cl)c1)C(=O)N1CCC[C@H]1C(=O)N[C@H](C#N)CC(=O)O]

or a pharmaceutical salt thereof, in effective amount for ameliorating, preventing or treating coronavirus infection, coronavirus disease 2019 (COVID-19), Multisystem Inflammatory Syndrome in Children (MIS-C) associated with coronavirus disease 2019 (COVID-19), Severe Acute Respiratory Syndrome (SARS), Middle East Respiratory Syndrome (MERS), pathogen-related respiratory distress syndrome, pathogen-related multi-organ failure, cytokine release syndrome, cytokine storm syndrome, or diseases associated with excessive activation of the canonic and non-canonic inflammasomes.

Advantageously, the inhibitor of inflammatory caspases of formula (V) is belnacasan. Belnacasan is an acetyl pro-drug with a reversible leaving group (aldehyde). Belnacasan, upon conversion in its active form VRT-043198 is a potent inhibitor of Caspase 1 (Ki =0.8 nm) and Caspase-4 (Ki<0.6 nM) which exhibits a more moderate effect on Caspase-8 (Ki=100 nM), weak on Caspase-9 (Ki=1 μM), and very low on Caspase-3 (Ki=16 μM) and Caspase-7 (Ki=21.5 μM). Belnacasan has been shown to be effective in cellular models of interleukin-1β release after LPS treatment.

In a particular embodiment, the coronavirus infection is selected among the group of human coronavirus 229E, human coronavirus OC43, Severe Acute Respiratory Syndrome coronavirus (SARS-CoV-1), Severe Acute Respiratory Syndrome coronavirus (SARS-CoV-2 or COVID-19), human Coronavirus NL63 (HCoV-NL63, New Haven coronavirus), human coronavirus HKU1; and Middle East respiratory syndrome coronavirus (MERS-CoV).

The present invention also relates to a pharmaceutical composition as defined above, for its use as a medicament. In particular, the present invention relates to a pharmaceutical composition comprising an inhibitor of inflammatory caspases in effective amount for ameliorating, preventing or treatingcoronavirus infection, coronavirus disease 2019 (COVID-19), Multisystem Inflammatory Syndrome in Children (MIS-C) associated with coronavirus disease 2019 (COVID-19), Severe Acute Respiratory Syndrome (SARS), Middle East Respiratory Syndrome (MERS), pathogen-related respiratory distress syndrome, pathogen-related multi-organ failure, cytokine release syndrome, cytokine storm syndrome, or diseases associated with excessive activation of the canonic and non-canonic inflammasomes.

The term “prevention” or “prophylaxis” or “preventative treatment” or “prophylactic treatment” or “preventing” comprises a treatment leading to the prevention of a disease as well as a treatment reducing and/or delaying the incidence of a disease or the risk of it occurring.

According to the invention, the pharmaceutical composition comprising an inhibitor of inflammatory caspases is particularly useful for preventing or reducing the deleterious cascade upstream of cytokine release and thus to avoid the caveat of inhibiting only one cytokine among many.

The term “treatment” or “curative treatment” or “treating” is defined as a treatment leading to a cure or a treatment which alleviates, improves and/or eliminates, reduces and/or stabilizes the symptoms of a disease or the suffering that it causes.

According to the invention, the pharmaceutical composition comprising an inhibitor of inflammatory caspases is particularly useful for treating coronavirus infection, coronavirus disease 2019 (COVID-19), Multisystem Inflammatory Syndrome in Children (MIS-C) associated with coronavirus disease 2019 (COVID-19), Severe Acute Respiratory Syndrome (SARS), Middle East Respiratory Syndrome (MERS), pathogen-related respiratory distress syndrome, pathogen-related multi-organ failure, cytokine release syndrome, cytokine storm syndrome, or diseases associated with excessive activation of the canonic and non-canonic inflammasomes.

Another object of the invention concerns a pharmaceutical composition comprising a pharmaceutically effective amount of the inhibitor of inflammatory caspases as defined above as an active substance and at least one pharmaceutically acceptable carrier.

Advantageously, the pharmaceutical composition comprising a pharmaceutically effective amount of the inhibitor of inflammatory caspases of the invention further comprising at least one pharmaceutically acceptable carrier.

The term “pharmaceutically acceptable carrier” refers to a non-toxic carrier that may be administered to a patient, together with the inhibitor of inflammatory caspases of this invention, and which does not destroy the pharmacological activity thereof.

Pharmaceutically acceptable carriers that may be used in the pharmaceutical composition of the invention include, but are not limited to, ion exchangers, alumina, aluminum stearate, lecithin, serum proteins such as human serum albumin, buffer substances such as phosphates, glycine, sorbic acid, potassium sorbate, partial glyceride mixtures of saturated vegetable fatty acids, water, salts or electrolytes such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

Also, the basic nitrogen-containing groups can be quaternized with such agents as lower alkyl halides, such as methyl, ethyl, propyl, and butyl chlorides, bromides and iodides; dialkyl sulfates, such as dimethyl, diethyl, dibutyl and diamyl sulfates; long chain halides such as decyl, lauryl, myristyl and stearyl chlorides, bromides and iodides; aralkyl halides, such as benzyl and phenethyl bromides and others. Water or oil-soluble or dispersible products are thereby obtained.

The inhibitor of inflammatory caspases utilized in the composition of this invention may also be modified by appending appropriate functionalities to enhance selective biological properties. Such modifications are known in the art and include those which increase biological penetration into a given biological system (e.g., blood, lymphatic system, or central nervous system), increase oral availability, increase solubility to allow administration by injection, alter metabolism and/or alter rate of excretion.

Advantageously, the pharmaceutical composition comprises a pharmaceutically effective amount of the inhibitor of inflammatory caspases as defined above or a pharmaceutical salt thereof, as an active substance.

Advantageously, the pharmaceutical composition comprising a therapeutically effective amount of the inhibitor of inflammatory caspases or a pharmaceutical salt thereof as defined above as an active substance is administered to a patient suffering from or suspected of suffering from coronavirus infection, cytokine release syndrome (CRS), or cytokine storm syndrome (CSS). Advantageously, he coronavirus infection is selected among the group of human coronavirus 229E, human coronavirus OC43, Severe Acute Respiratory Syndrome coronavirus (SARS-CoV-1), Severe Acute Respiratory Syndrome coronavirus (SARS-CoV-2 or COVID-19), human Coronavirus NL63 (HCoV-NL63, New Haven coronavirus), human coronavirus HKU1; and Middle East respiratory syndrome coronavirus (MERS-CoV).

In one embodiment the patient is a human. Advantageously, the patient can be a human adult, a human child or a human baby. Advantageously, the patient can have comorbidities or not. In another embodiment the patient is a non-human animal, e.g., a dog, cat, horse, cow, pig, sheep, goat or primate.

According to embodiments that involve administering to a patient in need of treatment a therapeutically effective amount of the inhibitor of inflammatory caspases as provided herein, “therapeutically effective” or “an amount effective” or “effective amount” or “pharmaceutically effective” denotes the amount of the inhibitor of inflammatory caspases needed to inhibit or reverse a disease condition (e.g., to prevent, to ameliorate or to treat coronavirus infection, coronavirus disease 2019 (COVID-19), Multisystem Inflammatory Syndrome in Children (MIS-C) associated with coronavirus disease 2019 (COVID-19), Severe Acute Respiratory Syndrome (SARS), Middle East Respiratory Syndrome (MERS), pathogen-related respiratory distress syndrome, pathogen-related multi-organ failure, cytokine release syndrome, cytokine storm syndrome, or diseases associated with excessive activation of the canonic and non-canonic inflammasomes). Determining a therapeutically effective amount specifically depends on such factors as toxicity and efficacy of the medicament. These factors will differ depending on other factors such as potency, relative bioavailability, patient body weight, severity of adverse side-effects and preferred mode of administration. Toxicity may be determined using methods well known in the art. Efficacy may be determined utilizing the same guidance. Efficacy, for example, can be measured by ELISA in order to determine the level of caspases 1, 4 and 5, IL1alpha, IL1beta, and IL-18. A pharmaceutically effective amount, therefore, is an amount that is deemed by the clinician to be toxicologically tolerable, yet efficacious.

Dosage may be adjusted appropriately to achieve desired drug (e.g., inhibitor of inflammatory caspases) levels, local or systemic, depending upon the mode of administration. In the event that the response in a subject is insufficient at such doses, even higher doses (or effective higher doses by a different, more localized delivery route) may be employed to the extent that patient tolerance permits. Multiple doses per day may also be employed to achieve appropriate systemic levels of antibodies. Appropriate systemic levels can be determined by, for example, measurement of the patient's peak or sustained plasma level of the drug. “Dose” and “dosage” are used interchangeably herein.

In some embodiments, the inhibitor of inflammatory caspases is administered to the patient at a dose of 300 mg to 2,400 mg per administration.

Advantageously, the inhibitor of inflammatory caspases is administered to the patient at a dose of 600 mg to about 1800 mg per administration.

Advantageously, the inhibitor of inflammatory caspases is administered to the patient at a dose of 900 mg per administration.

Typically, the pharmaceutical composition of this invention will be administered from about 1 to 5 times per day or alternatively, as a continuous infusion. Such administration can be used as a chronic or acute therapy. The amount of active substance that may be combined with the carrier materials to produce a single dosage form will vary depending upon the host treated and the particular mode of administration. A typical preparation will contain from about 5% to about 95% active substance (w/w). Preferably, such preparations contain from about 20% to about 80% active substance.

In an advantageous embodiment, the pharmaceutical composition of the invention further comprises a second active ingredient.

Advantageously, the second active ingredient is selected among N-Acetyl-cysteine, Fibrin-derived peptide B1315-42, Vitamin D, molnupiravir, SARS-CoV-2 protease inhibitors including PF-07321332 or PF-07304814, and an antibiotic or a combination of antibiotic.

Non-exhaustive examples of antibiotic can be selected among the group comprising ceftriaxone, spiramycin, amoxicillin, amoxicillin/clavulanic acid, gentamicin, netilmicin, piperacilin/tazobactam, amikacin, cefuroxime, penicillin, azithromycin, clarithromycin, erythromycin, doxycycline, cefotaxime, ampicillin, Ertapenem, cefepime, imipenem, meropenem, metronidazole, fluconazole, ciprofloxacin, levofloxacin, vancomycin, linezolid, moxifloxacin and gemifloxacin.

For instance, SARS-COV-1 or SARS-COV-2 infected individual and placed recently under artitifical ventilation may preferably receive a combinaition of intravenous Belnacasan (600 mg to 1200 mg) and cefotaxime (2 g/8 h), ceftriaxone (2 g/j), or amoxicilline or clavulanic acid (2 g/8 h). Alternatively, Belnacasan combined with betalactamin antipseudomonas and ciprofloxacin (400 mg/8h).

In a preferred embodiment, the invention provides a method of treating a patient, having one of the aforementioned diseases, comprising the step of administering to said patient a pharmaceutically acceptable composition described above. In this embodiment, if the patient is also administered second active ingredient, it may be delivered together with the pharmaceutical composition comprising the inhibitor of inflammatory caspases of this invention in a single dosage form, or, as a separate dosage form. When administered as a separate dosage form, the second active ingredient may be administered prior to, at the same time as, or following administration of a pharmaceutically acceptable composition comprising the inhibitor of inflammatory caspases of this invention.

When the composition of this invention comprises a second active ingredient, both the inhibitor of inflammatory caspases and the second active ingredient should be present at dosage levels of between about 10% to about 80% of the dosage normally administered in a monotherapy regime.

Upon improvement of a patient's condition, a maintenance dose of the inhibitor of inflammatory caspases, composition or combination of this invention may be administered, if necessary. Subsequently, the dosage or frequency of administration, or both, may be reduced, as a function of the symptoms, to a level at which the improved condition is retained. When the symptoms have been alleviated to the desired level, treatment may cease. Patients may, however, require intermittent treatment on a long-term basis upon any recurrence or disease symptoms.

In some embodiments, the compositions provided are employed for in vivo applications. Depending on the intended mode of administration in vivo the compositions used may be in the dosage form of solid, semi-solid or liquid such as, e.g., tablets, pills, powders, capsules, gels, ointments, liquids, suspensions, or the like. Preferably, the compositions are administered in unit dosage forms suitable for single administration of precise dosage amounts. The compositions may also include, depending on the formulation desired, at least one pharmaceutically acceptable carrier or diluent, which are defined as aqueous-based vehicles commonly used to formulate pharmaceutical compositions for animal or human administration. The diluent is selected so as not to affect the biological activity of the specific binding molecule or the fusion protein of interest. Examples of such diluents are distilled water, physiological saline, Ringer's solution, dextrose solution, and Hank's solution. The same diluents may be used to reconstitute a lyophilized recombinant protein of interest. In addition, the pharmaceutical composition may also include other medicinal agents, pharmaceutical agents, carriers, adjuvants, nontoxic, non-therapeutic, non-immunogenic stabilizers, etc. Effective amounts of such diluent or carrier are amounts which are effective to obtain a pharmaceutically acceptable formulation in terms of solubility of components, biological activity, etc. In some embodiments the compositions provided herein are sterile.

Administration during in vivo treatment may be by any routes, including oral, parenteral, intramuscular, intranasal, sublingual, intratracheal, inhalation, ocular, vaginal, and rectal. Intracapsular, intravenous, and intraperitoneal routes of administration may also be employed. The skilled artisan recognizes that the route of administration varies depending on the disorder to be treated. For example, the pharmaceutical composition of the invention may be administered to the patient via oral, parenteral or topical administration. In one embodiment, the pharmaceutical composition of the invention is administered oral, parenteral, intramuscular, intranasal, sublingual, intratracheal, inhalation, ocular, vaginal, and rectal route. In one embodiment, the pharmaceutical composition of the invention is in a form administrable by oral, parenteral, intramuscular, intranasal, sublingual, intratracheal, inhalation, ocular, vaginal, and rectal route. Advantageously, the pharmaceutical composition is in a suitable form for its administration by oral, intravenous, parenteral, intramuscular, intranasal, sublingual, intratracheal, inhalation, ocular, vaginal, and rectal route. In one embodiment, the pharmaceutical composition of the invention is administered by intravenous infusion.

The composition, when it is desirable to deliver them systemically, may be formulated for parenteral administration by injection, e.g., by bolus injection or continuous infusion. Formulations for injection may be presented in unit dosage form, e.g., in ampoules or in multi-dose containers, with an added preservative. The composition may take such forms as suspensions, solutions or emulsions in oily or aqueous vehicles, and may contain formulatory agents such as suspending, stabilizing and/or dispersing agents.

Pharmaceutical formulation for parenteral administration includes aqueous solutions of the active compositions in water soluble form. Additionally, suspensions of the active compositions may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compositions to allow for the preparation of highly concentrated solutions. Alternatively, the active compositions may be in powder form for constitution with a suitable vehicle, e.g., sterile pyrogen-free water, before use.

For oral administration, the pharmaceutical composition may take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate. The component or components may be chemically modified so that oral delivery of the antibodies is efficacious. Generally, the chemical modification contemplated is the attachment of at least one molecule to the antibodies, where said molecule permits (a) inhibition of proteolysis; and (b) uptake into the blood stream from the stomach or intestine. Also desired is the increase in overall stability of the antibodies and increase in circulation time in the body. Examples of such molecules include: polyethylene glycol, copolymers of ethylene glycol and propylene glycol, carboxymethyl cellulose, dextran, polyvinyl alcohol, polyvinyl pyrrolidone and polyproline. Other polymers that could be used are poly-1,3-dioxolane and poly-1,3,6-tioxocane. Preferred for pharmaceutical usage, as indicated above, are polyethylene glycol molecules. For oral composition, the location of release may be the stomach, the small intestine (the duodenum, the jejunum, or the ileum), or the large intestine. One skilled in the art has available formulations which will not dissolve in the stomach, yet will release the material in the duodenum or elsewhere in the intestine. Preferably, the release will avoid the deleterious effects of the stomach environment, either by protection of the antibody or by release of the biologically active material beyond the stomach environment, such as in the intestine.

For buccal administration, the composition may take the form of tablets or lozenges formulated in conventional manner.

For administration by inhalation, the composition for use according to the present disclosure may be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g. gelatin for use in an inhaler or insufflator may be formulated containing a powder mix of the compositions and a suitable powder base such as lactose or starch.

Also contemplated herein is pulmonary delivery. The composition can be delivered to the lungs of a mammal while inhaling and traverses across the lung epithelial lining to the blood stream. Contemplated for use in the practice of this disclosure are a wide range of mechanical devices designed for pulmonary delivery of therapeutic products, including but not limited to nebulizers, metered dose inhalers, and powder inhalers, all of which are familiar to those skilled in the art.

Nasal delivery of a pharmaceutical composition disclosed herein is also contemplated. Nasal delivery allows the passage of a pharmaceutical composition of the present disclosure to the blood stream directly after administering the therapeutic product to the nose, without the necessity for deposition of the product in the lung. Formulations for nasal delivery include those with dextran or cyclodextran.

The composition may also be formulated in rectal or vaginal composition such as suppositories or retention enemas, e.g., containing conventional suppository bases such as cocoa butter or other glycerides.

The pharmaceutical composition also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

Suitable liquid or solid pharmaceutical preparation forms are, for example, aqueous or saline solutions for inhalation, microencapsulated, encochleated, coated onto microscopic gold particles, contained in liposomes, nebulized, aerosols, pellets for implantation into the skin, or dried onto a sharp object to be scratched into the skin. The pharmaceutical compositions also include granules, powders, tablets, coated tablets, (micro)capsules, suppositories, syrups, emulsions, suspensions, creams, drops or preparations with protracted release of active compositions, in whose preparation excipients and additives and/or auxiliaries such as disintegrants, binders, coating agents, swelling agents, lubricants, flavorings, sweeteners or solubilizers are customarily used as described above. The pharmaceutical compositions are suitable for use in a variety of drug delivery systems.

In a particular advantageous embodiment, the present invention relates to pharmaceutical composition comprising an inhibitors of inflammatory caspases as defined above or a pharmaceutically acceptable salt thereof, as a unique active ingredient, in an effective amount for 40 ameliorating, preventing or treating coronavirus infection, coronavirus disease 2019 (COVID-19), Multisystem Inflammatory Syndrome in Children (MIS-C) associated with coronavirus disease 2019 (COVID-19), Severe Acute Respiratory Syndrome (SARS), Middle East Respiratory Syndrome (MERS), pathogen-related respiratory distress syndrome, pathogen-related multi-organ failure, cytokine release syndrome, cytokine storm syndrome, or diseases associated with excessive activation of the canonic and non-canonic inflammasomes.

Advantageously, the present invention relates to a pharmaceutical composition comprising an inhibitors of inflammatory caspases as defined above or a pharmaceutically acceptable salt thereof, as a unique active ingredient, in an effective amount forits use in ameliorating, preventing or treating coronavirus infection, coronavirus disease 2019 (COVID-19), Multisystem Inflammatory Syndrome in Children (MIS-C) associated with coronavirus disease 2019 (COVID-19), Severe Acute Respiratory Syndrome (SARS), Middle East Respiratory Syndrome (MERS), pathogen-related respiratory distress syndrome, pathogen-related multi-organ failure, cytokine release syndrome, cytokine storm syndrome, or diseases associated with excessive activation of the canonic and non-canonic inflammasomes.

Advantageously, the pharmaceutical composition comprising an inhibitor of inflammatory caspases or a pharmaceutically acceptable salt thereof as defined above as a unique active substance, is administered to a subject suffering from or suspected of suffering from coronavirus infection, coronavirus disease 2019 (COVID-19), Multisystem Inflammatory Syndrome in Children (MIS-C) associated with coronavirus disease 2019 (COVID-19), Severe Acute Respiratory Syndrome (SARS), Middle East Respiratory Syndrome (MERS), pathogen-related respiratory distress syndrome, pathogen-related multi-organ failure, cytokine release syndrome, cytokine storm syndrome, or diseases associated with excessive activation of the canonic and non-canonic inflammasomes.

By “administered” or “administration” is meant the injection or the delivery to the patient of the pharmaceutical composition according to the invention.

Advantageously, the pharmaceutical composition comprises a therapeutically effective amount of inhibitor of inflammatory caspases of formula (I)

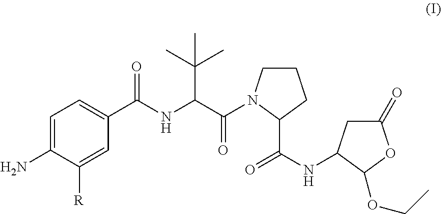

wherein R is H, OH, CH₃ or a halogen, or a pharmaceutically acceptable salt thereof as a unique active substance, and is administered to a subject suffering from or suspected of suffering from viral infections and disorders associated to the viral infections, coronavirus infection, coronavirus disease 2019 (COVID-19), Multisystem Inflammatory Syndrome in Children (MIS-C) associated with coronavirus disease 2019 (COVID-19), Severe Acute Respiratory Syndrome (SARS), Middle East Respiratory Syndrome (MERS), pathogen-related respiratory distress syndrome, pathogen-related multi-organ failure, cytokine release syndrome, cytokine storm syndrome, or diseases associated with excessive activation of the canonic and non-canonic inflammasomes.

Advantageously, the pharmaceutical composition comprises a therapeutically effective amount of inhibitor of inflammatory caspases of formula (II)

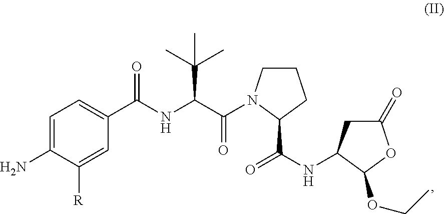

wherein R is H, OH, CH₃ or a halogen or a pharmaceutically acceptable salt thereof as a unique active substance, and is administered to a subject suffering from or suspected of suffering from viral infections and disorders associated to the viral infections, coronavirus infection, coronavirus disease 2019 (COVID-19), Multisystem Inflammatory Syndrome in Children (MIS-C) associated with coronavirus disease 2019 (COVID-19), Severe Acute Respiratory Syndrome (SARS), Middle East Respiratory Syndrome (MERS), pathogen-related respiratory distress syndrome, pathogen-related multi-organ failure, cytokine release syndrome, cytokine storm syndrome, or diseases associated with excessive activation of the canonic and non-canonic inflammasomes.

Advantageously, the pharmaceutical composition comprises a therapeutically effective amount of inhibitor of inflammatory caspases of formula (III)

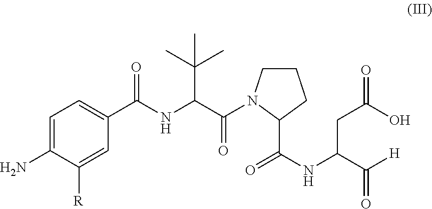

wherein R is H, OH, CH₃ or a halogen or a pharmaceutically acceptable salt thereof as a unique active substance, and is administered to a subject suffering from or suspected of suffering from viral infections and disorders associated to the viral infections, coronavirus infection, coronavirus disease 2019 (COVID-19), Multisystem Inflammatory Syndrome in Children (MIS-C) associated with coronavirus disease 2019 (COVID-19), Severe Acute Respiratory Syndrome (SARS), Middle East Respiratory Syndrome (MERS), pathogen-related respiratory distress syndrome, pathogen-related multi-organ failure, cytokine release syndrome, cytokine storm syndrome, or diseases associated with excessive activation of the canonic and non-canonic inflammasomes.

Advantageously, the pharmaceutical composition comprises a therapeutically effective amount of inhibitor of inflammatory caspases of formula (IV)

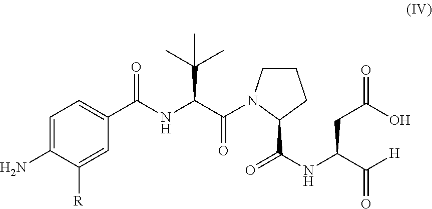

wherein R is H, OH, CH₃ or a halogen or a pharmaceutically acceptable salt thereof as a unique active substance, and is administered to a subject suffering from or suspected of suffering from viral infections and disorders associated to the viral infections, coronavirus infection, coronavirus disease 2019 (COVID-19), Multisystem Inflammatory Syndrome in Children (MIS-C) associated with coronavirus disease 2019 (COVID-19), Severe Acute Respiratory Syndrome (SARS), Middle East Respiratory Syndrome (MERS), pathogen-related respiratory distress syndrome, pathogen-related multi-organ failure, cytokine release syndrome, cytokine storm syndrome, or diseases associated with excessive activation of the canonic and non-canonic inflammasomes.

Advantageously, the pharmaceutical composition comprises a therapeutically effective amount of inhibitor of inflammatory caspases of formula (V):

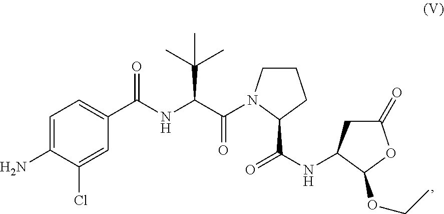

wherein R is H, OH, CH₃ or a halogen or a pharmaceutically acceptable salt thereof as a unique active substance, and is administered to a subject suffering from or suspected of suffering from preventing coronavirus infection, coronavirus disease 2019 (COVID-19), Multisystem Inflammatory Syndrome in Children (MIS-C) associated with coronavirus disease 2019 (COVID-19), Severe Acute Respiratory Syndrome (SARS), Middle East Respiratory Syndrome (MERS), pathogen-related respiratory distress syndrome, pathogen-related multi-organ failure, cytokine release syndrome, cytokine storm syndrome, or diseases associated with excessive activation of the canonic and non-canonic inflammasomes.

Advantageously, the inhibitor of inflammatory caspases of formula (V) is belnacasan.

Advantageously, the pharmaceutical composition comprises a therapeutically effective amount of inhibitor of inflammatory caspases of formula (VI):

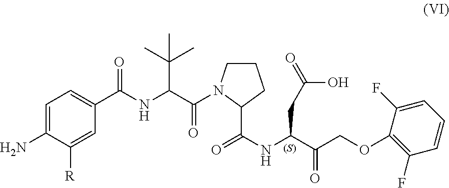

wherein R is H, OH, CH₃ or a halogen, or a pharmaceutical salt thereof as a unique active substance, and is administered to a subject suffering from or suspected of suffering from preventing coronavirus infection, coronavirus disease 2019 (COVID-19), Multisystem Inflammatory Syndrome in Children (MIS-C) associated with coronavirus disease 2019 (COVID-19), Severe Acute Respiratory Syndrome (SARS), Middle East Respiratory Syndrome (MERS), pathogen-related respiratory distress syndrome, pathogen-related multi-organ failure, cytokine release syndrome, cytokine storm syndrome, or diseases associated with excessive activation of the canonic and non-canonic inflammasomes.

Advantageously, the pharmaceutical composition comprises a therapeutically effective amount of inhibitor of inflammatory caspases of formula (VII):

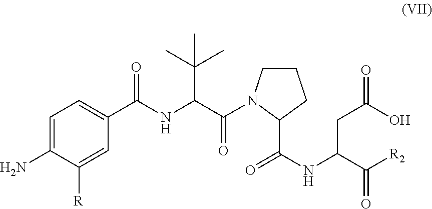

wherein R is H, OH, CH₃ or a halogen, or a pharmaceutical salt, and wherein R2 is

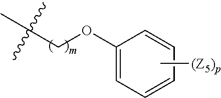

in which m is 0, 1 or 2
Z is a halogen,
p is 1, 2, 3, 4, or 5;
or a pharmaceutically acceptable salt thereof,
as a unique active substance, and is administered to a subject suffering from or suspected of suffering from preventing coronavirus infection, coronavirus disease 2019 (COVID-19), Multisystem Inflammatory Syndrome in Children (MIS-C) associated with coronavirus disease 2019 (COVID-19), Severe Acute Respiratory Syndrome (SARS), Middle East Respiratory Syndrome (MERS), pathogen-related respiratory distress syndrome, pathogen-related multi-organ failure, cytokine release syndrome, cytokine storm syndrome, or diseases associated with excessive activation of the canonic and non-canonic inflammasomes.

Advantageously, the pharmaceutical composition comprises a therapeutically effective amount of inhibitor of inflammatory caspases of formula (VIII):

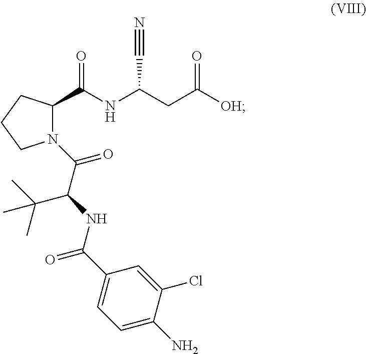 [SMILES: CC(C)(C)[C@H](NC(=O)c1ccc(N)c(Cl)c1)C(=O)N1CCC[C@H]1C(=O)N[C@H](C#N)CC(=O)O]

or a pharmaceutically acceptable salt thereof as a unique active substance, and is administered to a subject suffering from or suspected of suffering from preventing coronavirus infection, coronavirus disease 2019 (COVID-19), Multisystem Inflammatory Syndrome in Children (MIS-C) associated with coronavirus disease 2019 (COVID-19), Severe Acute Respiratory Syndrome (SARS), Middle East Respiratory Syndrome (MERS), pathogen-related respiratory distress syndrome, pathogen-related multi-organ failure, cytokine release syndrome, cytokine storm syndrome, or diseases associated with excessive activation of the canonic and non-canonic inflammasomes.

In advantageous embodiments, the pharmaceutical composition comprising an inhibitor of inflammatory caspases as defined above or a pharmaceutically acceptable salt thereof, as a unique active ingredient, is administered to the patient at a dose of 2 to 200 mg/kg of body weight/day. Advantageously, the pharmaceutical composition comprising an inhibitor of inflammatory caspases as defined above or a pharmaceutically acceptable salt thereof, as a unique active ingredient, is administered to the patient at a dose of 6 to 100 mg/kg of body weight/day. Advantageously, the pharmaceutical composition comprising an inhibitor of inflammatory caspases as defined above or a pharmaceutically acceptable salt thereof, as a unique active ingredient, is administered to the patient at a dose of 25 to 75 mg/kg of body weight/day.

Another object of the invention relates to a method for ameliorating, treating or preventing coronavirus infection, coronavirus disease 2019 (COVID-19), Multisystem Inflammatory Syndrome in Children (MIS-C) associated with coronavirus disease 2019 (COVID-19), Severe Acute Respiratory Syndrome (SARS), Middle East Respiratory Syndrome (MERS), pathogen-related respiratory distress syndrome, pathogen-related multi-organ failure, cytokine release syndrome, cytokine storm syndrome, or diseases associated with excessive activation of the canonic and non-canonic inflammasomes comprising the inhibitor of inflammatory caspases or a pharmaceutical salt thereof as defined above.

Advantageously, the method for ameliorating, treating or preventing coronavirus infection, coronavirus disease 2019 (COVID-19), Multisystem Inflammatory Syndrome in Children (MIS-C) associated with coronavirus disease 2019 (COVID-19), Severe Acute Respiratory Syndrome (SARS), Middle East Respiratory Syndrome (MERS), pathogen-related respiratory distress syndrome, pathogen-related multi-organ failure, cytokine release syndrome, cytokine storm syndrome, or diseases associated with excessive activation of the canonic and non-canonic inflammasomes, comprising administering to the patient the pharmaceutical composition comprising the inhibitor of inflammatory caspases of formula (I)

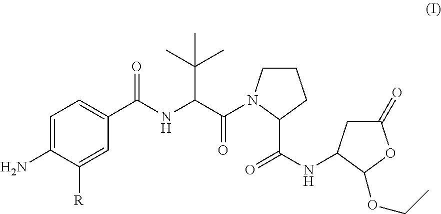

wherein R is H, OH, CH₃ or a halogen,or a pharmaceutical salt thereof as defined above.

Advantageously, the method for ameliorating, treating or preventing coronavirus infection, coronavirus disease 2019 (COVID-19), Multisystem Inflammatory Syndrome in Children (MIS-C) associated with coronavirus disease 2019 (COVID-19), Severe Acute Respiratory Syndrome (SARS), Middle East Respiratory Syndrome (MERS), pathogen-related respiratory distress syndrome, pathogen-related multi-organ failure, cytokine release syndrome, cytokine storm syndrome, or diseases associated with excessive activation of the canonic and non-canonic inflammasomes, comprising administering to the patient the pharmaceutical composition comprising the inhibitor of inflammatory caspases of formula (II)

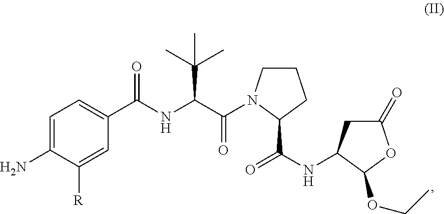

wherein R is H, OH, CH₃ or a halogen,or a pharmaceutical salt thereof as defined above.

Advantageously, the method for ameliorating, treating or preventing coronavirus infection, coronavirus disease 2019 (COVID-19), Multisystem Inflammatory Syndrome in Children (MIS-C) associated with coronavirus disease 2019 (COVID-19), Severe Acute Respiratory Syndrome (SARS), Middle East Respiratory Syndrome (MERS), pathogen-related respiratory distress syndrome, pathogen-related multi-organ failure, cytokine release syndrome, cytokine storm syndrome, or diseases associated with excessive activation of the canonic and non-canonic inflammasomes, comprising administering to the patient the pharmaceutical composition comprising the inhibitor of inflammatory caspases of formula (III)

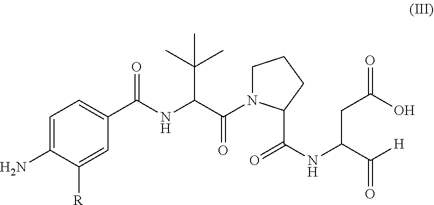

wherein R is H, OH, CH₃ or a halogen,or a pharmaceutical salt thereof as defined above.

Advantageously, the method for ameliorating, treating or preventing coronavirus infection, coronavirus disease 2019 (COVID-19), Multisystem Inflammatory Syndrome in Children (MIS-C) associated with coronavirus disease 2019 (COVID-19), Severe Acute Respiratory Syndrome (SARS), Middle East Respiratory Syndrome (MERS), pathogen-related respiratory distress syndrome, pathogen-related multi-organ failure, cytokine release syndrome, cytokine storm syndrome, or diseases associated with excessive activation of the canonic and non-canonic inflammasomes, comprising administering to the patient the pharmaceutical composition comprising the inhibitor of inflammatory caspases of formula (IV)

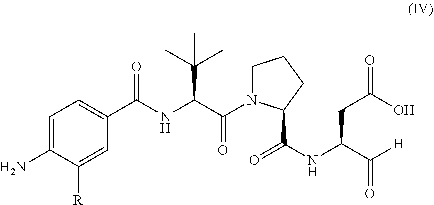

wherein R is H, OH, CH₃ or a halogen, or a pharmaceutical salt thereof as defined above.

Advantageously, the method for ameliorating, treating or preventing coronavirus infection, coronavirus disease 2019 (COVID-19), Multisystem Inflammatory Syndrome in Children (MIS-C) associated with coronavirus disease 2019 (COVID-19), Severe Acute Respiratory Syndrome (SARS), Middle East Respiratory Syndrome (MERS), pathogen-related respiratory distress syndrome, pathogen-related multi-organ failure, cytokine release syndrome, cytokine storm syndrome, or diseases associated with excessive activation of the canonic and non-canonic inflammasomes, comprising administering to the patient the pharmaceutical composition comprising the inhibitor of inflammatory caspases of formula (V)

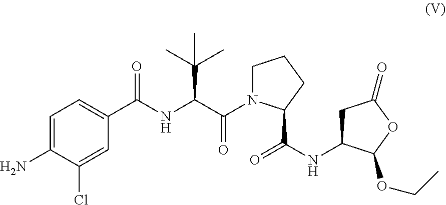

wherein R is H, OH, CH₃ or a halogen,or a pharmaceutical salt thereof as defined above.

Advantageously, the inhibitor of inflammatory caspases of formula (V) is belnacasan.

Advantageously, the method for ameliorating, treating or preventing coronavirus infection, coronavirus disease 2019 (COVID-19), Multisystem Inflammatory Syndrome in Children (MIS-C) associated with coronavirus disease 2019 (COVID-19), Severe Acute Respiratory Syndrome (SARS), Middle East Respiratory Syndrome (MERS), pathogen-related respiratory distress syndrome, pathogen-related multi-organ failure, cytokine release syndrome, cytokine storm syndrome, or diseases associated with excessive activation of the canonic and non-canonic inflammasomes, comprising administering to the patient the pharmaceutical composition comprising the inhibitor of inflammatory caspases of formula (VI):

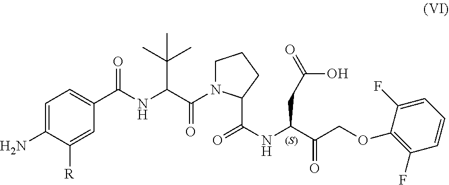

wherein R is H, OH, CH₃ or a halogen, or a pharmaceutical salt as defined above.

Advantageously, the method for ameliorating, treating or preventing coronavirus infection, coronavirus disease 2019 (COVID-19), Multisystem Inflammatory Syndrome in Children (MIS-C) associated with coronavirus disease 2019 (COVID-19), Severe Acute Respiratory Syndrome (SARS), Middle East Respiratory Syndrome (MERS), pathogen-related respiratory distress syndrome, pathogen-related multi-organ failure, cytokine release syndrome, cytokine storm syndrome, or diseases associated with excessive activation of the canonic and non-canonic inflammasomes, comprising administering to the patient the pharmaceutical composition comprising the inhibitor of inflammatory caspases of formula (VII)

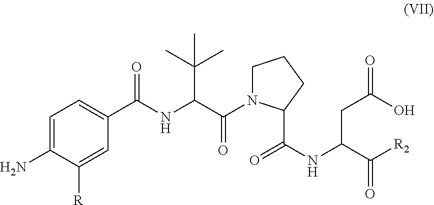

wherein R is H, OH, CH₃ or a halogen and wherein R2 is

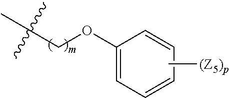

in which m is 0, 1 or 2
Z is a halogen,
p is 1, 2, 3, 4, or 5;
or a pharmaceutically acceptable salt thereof.

Advantageously, the method for ameliorating, treating or preventing coronavirus infection, coronavirus disease 2019 (COVID-19), Multisystem Inflammatory Syndrome in Children (MIS-C) associated with coronavirus disease 2019 (COVID-19), Severe Acute Respiratory Syndrome (SARS), Middle East Respiratory Syndrome (MERS), pathogen-related respiratory distress syndrome, pathogen-related multi-organ failure, cytokine release syndrome, cytokine storm syndrome, or diseases associated with excessive activation of the canonic and non-canonic inflammasomes, comprising administering to the patient the pharmaceutical composition comprising the inhibitor of inflammatory caspases of formula (VIII)

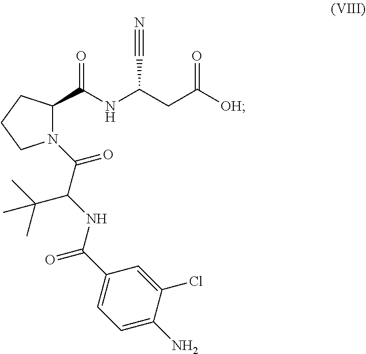 [SMILES: CC(C)(C)C(NC(=O)c1ccc(N)c(Cl)c1)C(=O)N1CCC[C@H]1C(=O)N[C@H](C#N)CC(=O)O]

or a pharmaceutically acceptable salt thereof as defined above

In one embodiment, the present invention relates to a method for ameliorating, treating or preventing coronavirus infection, comprising administering to the patient the pharmaceutical composition comprising the inhibitor of inflammatory caspases as defined above. Advantageously, the coronavirus infection is selected among the group of human coronavirus 229E, human coronavirus 0C43, Severe Acute Respiratory Syndrome coronavirus (SARS-CoV-1), Severe Acute Respiratory Syndrome coronavirus (SARS-CoV-2 or COVID-19), human Coronavirus NL63 (HCoV-NL63, New Haven coronavirus), human coronavirus HKU1; and Middle East respiratory syndrome coronavirus (MERS-CoV).

In one embodiment, the present invention relates to a method for ameliorating, treating or preventing cytokine release syndrome, comprising administering to the patient the pharmaceutical composition comprising the inhibitor of inflammatory caspases as defined above.

In one embodiment, the present invention relates to a method for ameliorating, treating or preventing cytokine storm syndrome, comprising administering to the patient the pharmaceutical composition comprising the inhibitor of inflammatory caspases as defined above.

In one embodiment, the method for ameliorating, treating or preventing coronavirus infection, coronavirus disease 2019 (COVID-19), Multisystem Inflammatory Syndrome in Children (MIS-C) associated with coronavirus disease 2019 (COVID-19), Severe Acute Respiratory Syndrome (SARS), Middle East Respiratory Syndrome (MERS), pathogen-related respiratory distress syndrome, pathogen-related multi-organ failure, cytokine release syndrome, cytokine storm syndrome, or diseases associated with excessive activation of the canonic and non-canonic inflammasomes, comprises administering to the patient the pharmaceutical composition comprising the inhibitor of inflammatory caspases as defined above, wherein the inhibitor of inflammatory caspases is administered to the patient at a dose of 300 mg to 2,400 mg per administration.

Advantageously, the inhibitor of inflammatory caspases is administered to the patient at a dose of 600 mg to about 1800 mg per administration.

Advantageously, the inhibitor of inflamatory caspases is administered to the patient at a dose of 900 mg per administration.

In one embodiment, the method for ameliorating, treating or preventing coronavirus infection, coronavirus disease 2019 (COVID-19), Multisystem Inflammatory Syndrome in Children (MIS-C) associated with coronavirus disease 2019 (COVID-19), Severe Acute Respiratory Syndrome (SARS), Middle East Respiratory Syndrome (MERS), pathogen-related respiratory distress syndrome, pathogen-related multi-organ failure, cytokine release syndrome, cytokine storm syndrome, or diseases associated with excessive activation of the canonic and non-canonic inflammasomes of the invention, comprises administering to the patient the pharmaceutical composition comprising the inhibitor of inflammatory caspases as defined above, further comprises at least one pharmaceutically acceptable carrier.

In one embodiment, the method for ameliorating, treating or preventing coronavirus infection, coronavirus disease 2019 (COVID-19), Multisystem Inflammatory Syndrome in Children (MIS-C) associated with coronavirus disease 2019 (COVID-19), Severe Acute Respiratory Syndrome (SARS), Middle East Respiratory Syndrome (MERS), pathogen-related respiratory distress syndrome, pathogen-related multi-organ failure, cytokine release syndrome, cytokine storm syndrome, or diseases associated with excessive activation of the canonic and non-canonic inflammasomes of the invention, comprises administering to the patient the pharmaceutical composition comprising the inhibitor of inflammatory caspases as defined above, further comprises a second active ingredient. Advantageously, the second active ingredient is selected among N-Acetyl-cystein, Fibrin-derived peptide B1315-42, Vitamin D, Molnupiravir, and an antibiotic. Non-exhaustive examples of antibiotic can be selected among the group comprising ceftriaxone, spiramycin, amoxicillin, amoxicillin/clavulanic acid, gentamicin, piperacilin/tazobactam, cefuroxime, penicillin, azithromycin, clarithromycin, erythromycin, doxycycline, cefotaxime, ampicillin, Ertapenem, cefepime, imipenem, meropenem, ciprofloxacin, levofloxacin, vancomycin, linezolid, moxifloxacin and gemifloxacin.

In one embodiment, the method for ameliorating, treating or preventing coronavirus infection, cytokine release syndrome (CRS), or cytokine storm syndrome (CSS) coronavirus infection, coronavirus disease 2019 (COVID-19), Multisystem Inflammatory Syndrome in Children (MIS-C) associated with coronavirus disease 2019 (COVID-19), Severe Acute Respiratory Syndrome (SARS), Middle East Respiratory Syndrome (MERS), pathogen-related respiratory distress syndrome, pathogen-related multi-organ failure, cytokine release syndrome, cytokine storm syndrome, or diseases associated with excessive activation of the canonic and non-canonic inflammasomes, of the invention, comprises administering to the patient the pharmaceutical composition comprising the inhibitor of inflammatory caspases as defined above.

In one embodiment, the patient is a patient infected by a coronavirus or has been admitted to hospital following a PCR detection of a pathogenic coronavirus. Advantageously, the patient is a patient infected by the SARS-CoV-2 or has been admitted to hospital following a PCR detection of SARS-CoV-2. Advantageously, the patient is a patient admitted to intensive care unit with elevated markers inflammation, low oxygen levels, or acute respiratory distress.

In one embodiment, the method for ameliorating, treating or preventing coronavirus infection, coronavirus disease 2019 (COVID-19), Multisystem Inflammatory Syndrome in Children (MIS-C) associated with coronavirus disease 2019 (COVID-19), Severe Acute Respiratory Syndrome (SARS), Middle East Respiratory Syndrome (MERS), pathogen-related respiratory distress syndrome, pathogen-related multi-organ failure, cytokine release syndrome, cytokine storm syndrome, or diseases associated with excessive activation of the canonic and non-canonic inflammasomes, comprises administering to the patient the pharmaceutical composition comprising the inhibitor of inflammatory caspases as defined above, the pharmaceutical composition of the invention being administered oral, parenteral, intramuscular, intranasal, sublingual, intratracheal, inhalation, ocular, vaginal, and rectal route.

In one embodiment, the method for ameliorating, treating or preventing coronavirus infection, coronavirus disease 2019 (COVID-19), Multisystem Inflammatory Syndrome in Children (MIS-C) associated with coronavirus disease 2019 (COVID-19), Severe Acute Respiratory Syndrome (SARS), Middle East Respiratory Syndrome (MERS), pathogen-related respiratory distress syndrome, pathogen-related multi-organ failure, cytokine release syndrome, cytokine storm syndrome, or diseases associated with excessive activation of the canonic and non-canonic inflammasomes of the invention, comprises administering to the patient the pharmaceutical composition comprising the inhibitor of inflammatory caspases as defined above, wherein the pharmaceutical composition is administered to the patient at a dose of 2 to 200 mg/kg of body weight/day. Advantageously, the pharmaceutical composition is administered to the patient at a dose of 6 to 100 mg/kg of body weight/day. Advantageously, the pharmaceutical composition is administered to the patient at a dose of 25 to 75 mg/kg of body weight/day.

In a particular advantageous embodiment, the present invention relates to the use of a pharmaceutical composition comprising a therapeutically effective amount of inhibitor of inflammatory caspases or a pharmaceutical salt thereof as defined above, as an active substance, in the manufacture of a medicinal product intended for ameliorating, treating or preventing coronavirus infection, coronavirus disease 2019 (COVID-19), Multisystem Inflammatory Syndrome in Children (MIS-C) associated with coronavirus disease 2019 (COVID-19), Severe Acute Respiratory Syndrome (SARS), Middle East Respiratory Syndrome (MERS), pathogen-related respiratory distress syndrome, pathogen-related multi-organ failure, cytokine release syndrome, cytokine storm syndrome, or diseases associated with excessive activation of the canonic and non-canonic inflammasomes.

In a particular advantageous embodiment, the present invention relates to a pharmaceutical composition comprising a therapeutically effective amount of inhibitor of inflammatory caspases or a pharmaceutical salt thereof as defined above, as an active substance, for its use in the amelioration, the prevention or the treatment of coronavirus infection, coronavirus disease 2019 (COVID-19), Multisystem Inflammatory Syndrome in Children (MIS-C) associated with coronavirus disease 2019 (COVID-19), Severe Acute Respiratory Syndrome (SARS), Middle East Respiratory Syndrome (MERS), pathogen-related respiratory distress syndrome, pathogen-related multi-organ failure, cytokine release syndrome, cytokine storm syndrome, or diseases associated with excessive activation of the canonic and non-canonic inflammasomes.

Accordingly, this invention also provides a method for reduction of Caspase-1 activation, IL-1β levels, IL-18 levels, or other biomarker in a patient having coronavirus infection, coronavirus disease 2019 (COVID-19), Multisystem Inflammatory Syndrome in Children (MIS-C) associated with coronavirus disease 2019 (COVID-19), Severe Acute Respiratory Syndrome (SARS), Middle East Respiratory Syndrome (MERS), pathogen-related respiratory distress syndrome, pathogen-related multi-organ failure, cytokine release syndrome, cytokine storm syndrome, or diseases associated with excessive activation of the canonic and non-canonic inflammasomes, comprising the step of administering to said patient the pharmaceutical composition of the invention. In one embodiment, the plasma concentration of the composition in the patient is about equal to or greater than an IC50 value taught according to this invention. In another embodiment, the plasma concentration of the composition in the patient is between about 0.2 μM to about 50 μM, or about 0.8 μM to about 6 μM.

In yet another embodiment, the reduction of IL-1β, IL-18 or other biomarker in a patient is measured by comparing (a) IL-1β, IL-18 or other biomarker concentrations in said patient before or after treatment with the composition of the invention to (b) activated Caspase-1, IL-1alpha, IL-1β, IL-18 or other biomarker concentrations in said patient during treatment with said the composition of the invention. According to this invention, the percent reduction of IL-1β, IL-18 or other biomarker in a patient is selected from the group consisting of (a) at least about 50% to about 100% reduction; (b) at least about 50% to about 90% reduction; and (c) at least about 60% to about 90% reduction.

Another object of the invention relates to a method for identifying a composition for treating coronavirus infection, coronavirus disease 2019 (COVID-19), Multisystem Inflammatory Syndrome in Children (MIS-C) associated with coronavirus disease 2019 (COVID-19), Severe Acute Respiratory Syndrome (SARS), Middle East Respiratory Syndrome (MERS), pathogen-related respiratory distress syndrome, pathogen-related multi-organ failure, cytokine release syndrome, cytokine storm syndrome, or diseases associated with excessive activation of the canonic and non-canonic inflammasomes in a patient comprising administering the pharmaceutical composition of the invention and comparing of biomarker for said coronavirus infection, coronavirus disease 2019 (COVID-19), Multisystem Inflammatory Syndrome in Children (MIS-C) associated with coronavirus disease 2019 (COVID-19), Severe Acute Respiratory Syndrome (SARS), Middle East Respiratory Syndrome (MERS), pathogen-related respiratory distress syndrome, pathogen-related multi-organ failure, cytokine release syndrome, cytokine storm syndrome, or diseases associated with excessive activation of the canonic and non-canonic inflammasomes, in the patient before and after treatment with the pharmaceutical composition of the invention.

The methods for identifying a compound or composition for treating coronavirus infection, cytokine release syndrome (CRS), or cytokine storm syndrome (CSS) according to this invention include methods for screening of a plurality of compounds or compositions for their ability to ameliorate the effects of coronavirus infection, coronavirus disease 2019 (COVID-19), Multisystem Inflammatory Syndrome in Children (MIS-C) associated with coronavirus disease 2019 (COVID-19), Severe Acute Respiratory Syndrome (SARS), Middle East Respiratory Syndrome (MERS), pathogen-related respiratory distress syndrome, pathogen-related multi-organ failure, cytokine release syndrome, cytokine storm syndrome, or diseases associated with excessive activation of the canonic and non-canonic inflammasomes. According to one embodiment of this invention, high throughput screening can be achieved by having cells in culture in a plurality of wells in a microtiter plate, adding a different compound or composition to each well and comparing the inflammatory caspases inhibition and/or IL-1(3 levels and/or activity in each cell culture to the levels or activity present in a cell culture in a control well. Controls that are useful for the comparison step according to this invention include cells or patients that have not been treated with a compound or composition and cells or subjects have been treated with a compound or composition that is known to have no effect on inflammatory caspases inhibition or activity. According to one embodiment of this invention, the high throughput screening is automated so that the steps including the addition of the cells to the plate up to the data collection and analysis after addition of the compound or composition are done by machine. Instruments that are useful in the comparison step of this invention, e.g., instruments that can detect labeled objects (e.g., radiolabelled, fluorescent or colored objects) or objects that are themselves detectable, are commercially available and/or known in the art. Accordingly, compounds and compositions according to this invention that are useful for treating coronavirus infection, coronavirus disease 2019 (COVID-19), Multisystem Inflammatory Syndrome in Children (MIS-C) associated with coronavirus disease 2019 (COVID-19), Severe Acute Respiratory Syndrome (SARS), Middle East Respiratory Syndrome (MERS), pathogen-related respiratory distress syndrome, pathogen-related multi-organ failure, cytokine release syndrome, cytokine storm syndrome, or diseases associated with excessive activation of the canonic and non-canonic inflammasomes disclosed herein can be quickly and efficiently screened.

In another embodiment of this invention, a method for identifying a composition for ameliorating, treating or preventing coronavirus infection in a patient comprises administering to said patient the pharmaceutical composition comprising the inhibitor of inflammatory caspases of the invention and comparing the inflammatory caspases inhibition in the patient before and after treatment with the pharmaceutical composition comprising the inhibitor of inflammatory caspases of the invention. In an alternate embodiment, this invention provides a method for identifying a composition for ameliorating, treating or preventing coronavirus infection in a patient which comprises administering to said patient the pharmaceutical composition comprising the inhibitor of inflammatory caspases of the invention and comparing a biomarker for coronavirus infection in said patient before and after treatment with said pharmaceutical composition comprising the inhibitor of inflammatory caspases of the invention.

In another embodiment of this invention, a method for identifying a composition for ameliorating, treating or preventing coronavirus infection in a patient comprises administering to said patient the pharmaceutical composition comprising the inhibitor of inflammatory caspases of the invention and comparing the inflammatory caspases inhibition in the patient before and after treatment with the pharmaceutical composition comprising the inhibitor of inflammatory caspases of the invention. In an alternate embodiment, this invention provides a method for identifying a composition for ameliorating, treating or preventing Multisystem Inflammatory Syndrome associated with coronavirus disease 2019 (COVID-19) in a patient which comprises administering to said patient the pharmaceutical composition comprising the inhibitor of inflammatory caspases of the invention and comparing a biomarker for coronavirus infection in said patient before and after treatment with said pharmaceutical composition comprising the inhibitor of inflammatory caspases of the invention.

In another embodiment of this invention, a method for identifying a composition for ameliorating, treating or preventing cytokine release syndrome in a patient comprises administering to said patient the pharmaceutical composition comprising the inhibitor of inflammatory caspases of the invention and comparing the inflammatory caspases inhibition in the patient before and after treatment with the pharmaceutical composition comprising the inhibitor of inflammatory caspases of the invention. In an alternate embodiment, this invention provides a method for identifying a composition for ameliorating, treating or preventing cytokine release syndrome in a patient which comprises administering to said patient the pharmaceutical composition comprising the inhibitor of inflammatory caspases of the invention and comparing a biomarker for coronavirus infection in said patient before and after treatment with said pharmaceutical composition comprising the inhibitor of inflammatory caspases of the invention.

In another embodiment of this invention, a method for identifying a composition for ameliorating, treating or preventing cytokine storm syndrome in a patient comprises administering to said patient the pharmaceutical composition comprising the inhibitor of inflammatory caspases of the invention and comparing the inflammatory caspases inhibition in the patient before and after treatment with the pharmaceutical composition comprising the inhibitor of inflammatory caspases of the invention. In an alternate embodiment, this invention provides a method for identifying a composition for ameliorating, treating or preventing cytokine storm syndrome in a patient which comprises administering to said patient the pharmaceutical composition comprising the inhibitor of inflammatory caspases of the invention and comparing a biomarker for coronavirus infection in said patient before and after treatment with said pharmaceutical composition comprising the inhibitor of inflammatory caspases of the invention.

The term “biomarker” is a physical, functional, or biochemical indicator, e.g., the presence of a particular metabolite, of a physiological or disease process. According to this invention, certain biomarkers of inflammation may be used to evaluate the response of patients having coronavirus infection, cytokine release syndrome (CRS), or cytokine storm syndrome (CSS) to inhibitor of inflammatory caspases. These inflammatory biomarkers include, but are not limited to IL-1β, activated caspase-1, cleaved caspase-1 (Casp1p20), cleaved IL-18, IL-6, IL-10, IL-4, LDH, C-reactive protein (CRP).

Another aspect of the invention concerns an inhibitor of inflammatory caspases of formula (VI):

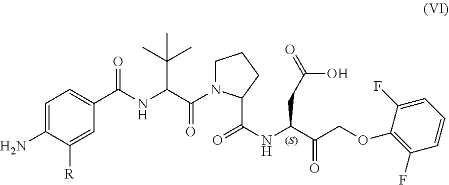

wherein R is H, OH, CH₃ or a halogen, or a pharmaceutical salt, in effective amount for ameliorating, preventing or treating coronavirus infection, coronavirus disease 2019 (COVID-19), Multisystem Inflammatory Syndrome in Children (MIS-C) associated with coronavirus disease 2019 (COVID-19), Severe Acute Respiratory Syndrome (SARS), Middle East Respiratory Syndrome (MERS), pathogen-related respiratory distress syndrome, pathogen-related multi-organ failure, cytokine release syndrome, cytokine storm syndrome, or diseases associated with excessive activation of the canonic and non-canonic inflammasomes.

Another aspect of the invention concerns an inhibitor of inflammatory caspases of formula (VI):

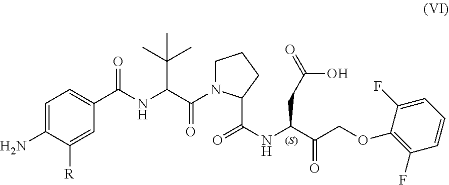

wherein R is H, OH, CH₃ or a halogen, or a pharmaceutical salt,in effective amount for its use in ameliorating, preventing or treating coronavirus infection, coronavirus disease 2019 (COVID-19), Multisystem Inflammatory Syndrome in Children (MIS-C) associated with coronavirus disease 2019 (COVID-19), Severe Acute Respiratory Syndrome (SARS), Middle East Respiratory Syndrome (MERS), pathogen-related respiratory distress syndrome, pathogen-related multi-organ failure, cytokine release syndrome, cytokine storm syndrome, or diseases associated with excessive activation of the canonic and non-canonic inflammasomes.

Another aspect of the invention concerns an inhibitor of inflammatory caspases of formula (VII)

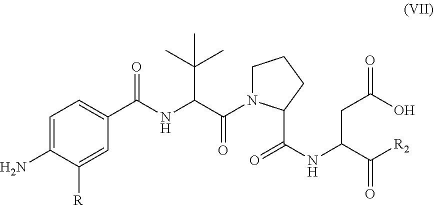

wherein R is H, OH, CH₃ or a halogen and wherein R2 is

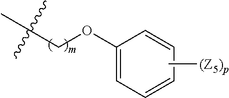

In which m is 0, 1 or 2

Z is a halogen,
p is 1, 2, 3, 4, or 5;
or a pharmaceutical salt thereof, in effective amount for ameliorating, preventing or treating coronavirus infection, coronavirus disease 2019 (COVID-19), Multisystem Inflammatory Syndrome in Children (MIS-C) associated with coronavirus disease 2019 (COVID-19), Severe Acute Respiratory Syndrome (SARS), Middle East Respiratory Syndrome (MERS), pathogen-related respiratory distress syndrome, pathogen-related multi-organ failure, cytokine release syndrome, cytokine storm syndrome, or diseases associated with excessive activation of the canonic and non-canonic inflammasomes.

Another aspect of the invention concerns an inhibitor of inflammatory caspases of formula (VII) :

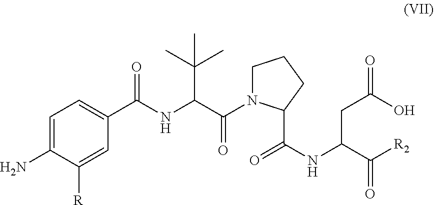

wherein R is H, OH, CH₃ or a halogen, and wherein R2 is

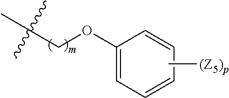

In which m is 0, 1 or 2

Z is a halogen,
p is 1, 2, 3, 4, or 5;
or a pharmaceutical salt thereof,
in effective amount for its use in ameliorating, preventing or treating coronavirus infection, coronavirus disease 2019 (COVID-19), Multisystem Inflammatory Syndrome in Children (MIS-C) associated with coronavirus disease 2019 (COVID-19), Severe Acute Respiratory Syndrome (SARS), Middle East Respiratory Syndrome (MERS), pathogen-related respiratory distress syndrome, pathogen-related multi-organ failure, cytokine release syndrome, cytokine storm syndrome, or diseases associated with excessive activation of the canonic and non-canonic inflammasomes.

Another aspect of the invention concerns an inhibitor of inflammatory caspases of formula (VIII):

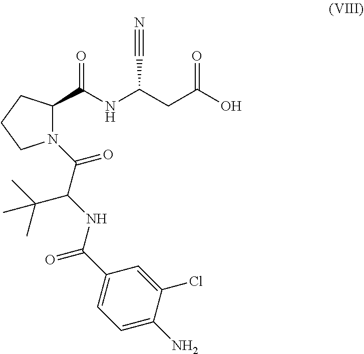 [SMILES: CC(C)(C)C(NC(=O)c1ccc(N)c(Cl)c1)C(=O)N1CCC[C@H]1C(=O)N[C@H](C#N)CC(=O)O]

or a pharmaceutical salt, in effective amount for ameliorating, preventing or treating coronavirus infection, coronavirus disease 2019 (COVID-19), Multisystem Inflammatory Syndrome in Children (MIS-C) associated with coronavirus disease 2019 (COVID-19), Severe Acute Respiratory Syndrome (SARS), Middle East Respiratory Syndrome (MERS), pathogen-related respiratory distress syndrome, pathogen-related multi-organ failure, cytokine release syndrome, cytokine storm syndrome, or diseases associated with excessive activation of the canonic and non-canonic inflammasomes.

Another aspect of the invention concerns an inhibitor of inflammatory caspases of formula (VIII):

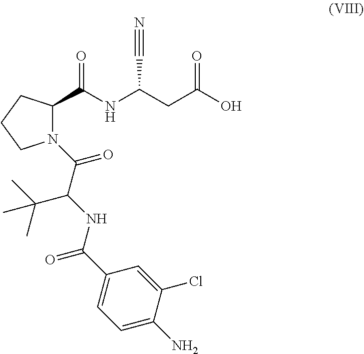 [SMILES: CC(C)(C)C(NC(=O)c1ccc(N)c(Cl)c1)C(=O)N1CCC[C@H]1C(=O)N[C@H](C#N)CC(=O)O]

or a pharmaceutical salt, in effective amount for its use in ameliorating, preventing or treating coronavirus infection, coronavirus disease 2019 (COVID-19), Multisystem Inflammatory Syndrome in Children (MIS-C) associated with coronavirus disease 2019 (COVID-19), Severe Acute Respiratory Syndrome (SARS), Middle East Respiratory Syndrome (MERS), pathogen-related respiratory distress syndrome, pathogen-related multi-organ failure, cytokine release syndrome, cytokine storm syndrome, or diseases associated with excessive activation of the canonic and non-canonic inflammasomes.

### EXAMPLES

### Example 1

### Cytokine Production in COVID-19 Patients

1/ COVID-19 Patients

Patients with COVID-19 are classified according to their clinical manifestations in: i) mild cases: the clinical symptoms are mild and no pneumonia manifestations can be found in imaging; ii) moderate cases: patients have symptoms such as fever and respiratory tract symptoms, etc. and pneumonia manifestations can be seen in imaging; iii) severe cases: adults who meet any of the following criteria: respiratory rate >30 breaths/min; oxygen saturations; 93% at a rest state; arterial partial pressure of oxygen (PaO2)/oxygen concentration (FiO2)<300 mm Hg. Belnacasan or coumpounds inhibiting Caspase-1, Caspase-4 and/or Caspase-5 may be used to inhibit pathogenic progression from mild/moderate to severe cases. Alternatively they may be used to treat severe cases.

2/ Peripheral blood mononuclear cells isolation

Whole blood is collected from healthy donors or from patients in tubes containing EDTA (BD Vacutainer CP 216 TTM), according to the manufacturer's instructions. The material is centrifuged at 400×g for 10 minutes at room temperature. Then, the plasma is discarded and the cell pellet is resuspended in PBS 1× pH 7.4 (GIBCO, BRL). The cells are applied to the Ficoll-PaqueTM gradient column (GE Healthcare Biosciences AB, Uppsala, Sweden). Then, they the mononuclear fraction is carefully collected and centrifuged at 640 g for 30 minutes at room temperature to obtain the purified mononuclear fraction, which is transferred to a new tube. The cells are washedwashed, and the pellet is resuspended in RPMI for the subsequent analysis.

3/ Purification of Monocytes from Healthy Donors and Differentiation into Macrophages

The PBMCs are quantified and the monocytes (CD14+ cells) are purified using positive selection with magnetic nanoparticles (BD). Briefly, PBMCs are labeled with BD IMag™ Anti-human CD14 Magnetic Particles. The cells are transferred to a 48-well culture plate and placed over a magnetic field. Labeled cells migrate toward the magnet (positive fraction) whereas unlabeled cells are drawn off (negative fraction). The plate is then removed from the magnetic field for resuspension of the positive fraction. The separation is repeated twice to increase the purity of the positive fraction. The CD14+ monocytes resulting cells from this process are used for experiments or cultured in RPMI 1640 (GIBCO, BRL) containing 10% SFB and 50 ng/mL GM-CSF (R&D Systems) for 7 days for differentiation into macrophages.

4/ Virus Stock Production and In Vitro Infection

SARS-CoV-2 strain are isolated from COVID-19 patients. Viral stocks are propagated under BSL3 conditions in Vero E6 cells, cultured in Dulbecco minimal essential medium (DMEM) supplemented with heat-inactivated fetal bovine serum (10%) and antibiotics/antimycotics (Penicillin 10,000 U/mL; Streptomycin 10,000 μg/mL). For preparation of viral stocks, Vero cells are infected in the presence of trypsin-TPCK (1 μg/μL) for 48 hours at 37° C. in a 5% CO2 atmosphere. When the virus induces cytopathic effects, the cells are harvested and centrifuged (10.000 g). The supernatant are stored at −80° C., and the virus titration is performed in Vero cells using standard limiting dilution to confirm the 50% tissue culture infectious dose (TCID50). For human cells infections, 2×105 purified human monocytes or monocyte derived macrophages are plated in 48 well plates, and infected with SARS-CoV-2 at Multiplicity of Infection (MOI) of MOI 0.2, MOI 1, and MOI 5. After 2 hours of viral infection, the cells are washed with PBS1×, and a new medium (RPMI 10% FBS without Fenol Red) is added. Cells are incubated for 24h at 37° C. in the presence of 5% CO2 atmosphere. After incubation, cells are processed for immunofluorescence assays and the supernatant was collected for determination of viral loads, cytokine production and LDH quantification.

5/ RT-PCR for SARS-CoV-2

Detection of SARS-CoV-2 is performed according to published protocols:

Corman, V. M. et al. Detection of 2019 novel coronavirus (2019-nCoV) by real-time RT-PCR. Euro surveillance: European communicable disease bulletin 2020, 25:469.

Nalla, A. K. et al. Comparative Performance of SARS-CoV-2 Detection Assays Using Seven Different Primer-Probe Sets and One Assay Kit. Journal of clinical microbiology 2020, 58, doi:10.1128/JCM.00557-20 (2020).

6/ Evaluation of active caspase-1 activity and LDH release in cultured cells

For LDH determination, 2×105 human CD14+ cells or human monocyte derived macrophages are plated on 48-well plates in RPMI 10% FBS and incubated overnight. In the following day, cells are infected with SARS-CoV-2 using MOI 0.2, MOI 1, and MOI 5 in RPMI without Phenol Red (3.5 g/L HEPES, 2 g/L NaHCO3, 10.4 g/L RPMI without Phenol Red, 1% glutamine, pH 7.2) and incubated for 24 h. The supernatant is collected and LDH release is measured using CytoTox 96® Non-Radioactive Cytotoxicity Assay (Promega, Winsconsin, USA) following the manufacturer's instructions. To evaluate caspase-1 activation, 5×105 PBMC from COVID-19 patients or healthy donors are centrifuged (400 g, 10 minutes) and cells are labeled for 30 minutes with the FLICA carboxyfluorescein reagent (FAM-YVAD-FMK, Immunochemistry Technologies, LLC), as recommended by the manufacturer. The cells are then washed two times with PBS 1×, and fixed with fixative reagent provided by the manufacturer. Acquisition is performed in fixed cells by flow cytometry (BD AccuriTM C6) and then analyzed using the “Flowio” software (Tree Star, Ashland, OR, USA). To evaluate caspase-1 activity in supernatants, 2×105 PBMCs are plated in 96-wells plates, and incubated overnight. To measure caspase-1 activity, the supernatants are collected, and incubated with the Luciferin WEHD-substrate provided by the Caspase-Glo 1 Assay (Promega). After 1 hour incubation at room temperature, luminescence is measured using SpectraMax i3 system (Molecular Devices).

7/ Immunofluorescence Staining of Isolated Cells

For staining PBMCs from COVID-19 patients, a total of 5×105 PBMCs are plated in 8 wells chamber slides for 1 h in RPMI without FBS for cell adhesion before fixation. For staining cells infected in vitro, a total of 2×105 human monocytes or monocyte differentiated macrophages are plated in 24-wells plate containing coverslips and infected with SARS-CoV-2 at chosen MOI for 16 h. For fixation of the samples, tissue culture supernatants are removed and cells were fixed with 4% paraformaldehyde (PFA) for 20 minutes at room temperature. PFA is removed, cells are washed with PBS1×, and the coverslips or chambers are processed for immunofluorescence. Briefly, cells are blocked and permeabilized using PBS lx with goat serum and 0.05% saponin for 1h at room temperature. Primary antibodies mix of rabbit mAb anti-NLRP3 (Cell Signaling, 1:1000) are diluted in blocking solution and added to each chamber/coverslip. After 1 h of incubation the samples are washed with PBS 1× and secondary antibodies are added and incubated for 1h at room temperature. Secondary antibodies used are goat anti-rabbit 488 (Invitrogen, 1:3000) and goat anti-rabbit 594 (Life Technologies,1:3000). Slides are washed stained with DAPI (1mM) and mounted using ProLong (Invitrogen).

8/ Cytokine Quantification

Active caspase-1 (Casp1p20) and IL-18 levels are evaluated by ELISA assay (R&D Systems) in the serum from patients with COVID-19 or health donors following manufacturer's instructions. TNF-α, IL-2, IL-4, IL-6, IL-10, IFN-γ, and IL-17 are quantified in the serum from patients with COVID-19 or health donors using a human CBA cytokine kit (Th1/Th2/Th17 Cytokine Kit, BD Biosciences) following manufacturer's instructions. IL-1β in the tissue culture supernatants of human monocytes or macrophages cells infected with SARS-CoV-2 is quantified by ELISA (R&D Systems) following manufacturer's instructions.

9/ Cultures of THP-1 Lines

THP-1 cells come from a 1-year-old child with leukemia. It is a pre-monocytic line, characterized by a suspension of (non-adherent) cells with a well rounded shape. The cells used come from ATCC. The cells are cultured at a density of 0.4.106 /mL in T75 flasks kept upright in RPMI 1640 Medium, Glutamax, Supplement HEPES (ThermoFischer Scientific, #72400027) supplemented with 10% FCS (previously decomplemented and flitre) and 1% PS antibiotics. The cultures are maintained at 37° C. under a humid atmosphere with 5% CO2.

10/ Differentiation of Cells

The differentiation of THP-1 is induced by adding phorbol 12-myritate 13-acetate (PMA, 5 mg/mL stock in DMSO, InvivoGen, # tlrl-pma) to the medium. More precisely, the latter mimics the structure of diacylglycerol (DAG) which is an activator of protein Kinase C (PKC). Thus, it allows the stimulation of the expression of transcription factors including NF-kB, which are involved in macrophage differentiation (Park E. K. et al., 2007; Daigneault M. et al., 2010). After treatment with PMA the cells acquire a macrophages-like phenotype, that is, they become adherent and no longer divide. In practice, the cells are seeded on 24-well plates at a density of 300.103 cells/well, the PMA is then added for 24 hours at a concentration of 50 nM. The next day, the cells are washed gently with PBS and fresh complete medium is added, the cells are then left for 3 days. On the day of treatment, the cells are washed with PBS and placed in presence or not of 1 /mL of Lipopolysaccharide-EK Ultrapure-LPS- (Stock 1 mg/mL in ultrapure water, InvivoGen, #tlrl-peklps) for 6 hours. LPS activates macrophages via the NLRP3 inflammasome, a platform for activating CASP-1. The latter then allows the cleavage of pro-IL1beta into IL1beta in the medium. At the end of the treatment with LPS, the medium is removed and the cells are placed in the presence or absence of pharmacological agents for 1 hour. Then, nigericin at 5, 10 and 20 μM is added for 30, 60, and 120 minutes. The latter is an antibiotic that causes the release of K+ ions from the cell to increase the activation of NLRP3 to “boost” the system.

11/ Study of Inflammation and Cell Death

At the end of the treatment, the supernatants are recovered with a view to assays for the cytokine IL1β by ELISA (Human IL-1(3 ELISA MAX™ Deluxe, Biolegend, # 437005). In addition, once the supernatant has been collected, the cells are labeled with Hoescht 33342 (2 μg/mL) and propidium iodide (1 μg/mL) in order to assess the cytotoxicity of the treatment on the cells. The two agents are added respectively 10 and 5 minutes before the photo is taken.

12/ Reagents and Abs

The following primary Abs were used for immunoblotting: NLRP3 (Cell Signaling 13158, 1:1000), IL-113 (Cell Signaling 12703, 1:1000), GFP (Cell Signaling 2955, 1:1000), TFEB (Cell Signaling 4240, 1:1000), CHOP (Cell Signaling 2895, 1:1000), LC3A/B (Cell Signaling clone D3U4C, 1:1000) Histone H3 (Cell Signaling clone D1H2, 1:2000), Myc (EMD Millipore clone 9E10, 1:2000), Flag (Sigma F1804, 1:1000), ASC (Santa Cruz sc-22514, 1:1000), LAMP1 (Santa Cruz clone H3A4, 1:2000), GAPDH-HRP (ProteinTech HRP-60004, 1:5000), and actin-HRP (Sigma A3854, 1:10,000). Anti-mouse (Cell Signaling 7076) or anti-rabbit (Cell Signaling 7074) HRP-conjugated secondary antibodies were used for ECL based detection. Primary Abs for immunofluorescence were NLRP3 (Enzo AIX-804-819), ASC (Santa Cruz sc-22514), ERp72 (Cell Signaling D70D12), and TGN38 (Novus, NBP1-03495). Alexa 568 conjugated polyclonal anti-mouse (Thermo Fisher, A11004) and anti-rabbit (Thermo Fisher, A11011) were used as secondary Abs prior to fluorescent imaging. For immunoprecipitation experiments, we used GFP antibodies coupled to magnetic beads (MBL, D153-9) and Flag antibodies coupled to agarose (Sigma, A2220) to pull down tagged proteins prior to immunoblotting. Cyclosporin A (R&D Systems, 1101) and Z-VAD-FMK (Sigma, V116) were used at 10 μM overnight, and Bafilomycin A1 (Sigma, B1793) at 100 nM for 4 h.

13/ Cells, Plasmids, and siRNAs

THP-1, HEK293, A549, and HeLa cells are obtained from the American Type Culture Collection (ATCC) and maintained following ATCC's recommendations. THP-1 monocytes are differentiated into macrophages by treating with PMA (50 nM) for 3 h or alternatively 24 h. To generate stable THP-1 cell lines, constructs expressing 8b-GFP or GFP are transfected followed by G418 (200 μg/ml) selection for 6 weeks. GFP-positive cells are FACS sorted twice and expanded. PCR primers for cDNA generation are derived from the SARS-CoV accessory gene sequences in accession number NC_004718 (NCBI). Constructs expressing N and C terminal GFP-tagged SARS-CoV ORF8b are made using the pEGFP-N1 and C1 vectors (Clontech). PCR mutagenesis of GFP-8b was used to generate the V77K GFP-8b point mutant, and ORF8b-Flag was made by replacing the 8b-GFP c-terminal GFP with a 3× Flag tag. All constructs are verified by DNA sequencing. To construct the NLRP3 Ds-Red construct, the Ds-Red coding region is PCRed from the pDs-Red-Monomer fluorescent vector (Clontech) and inserted into Flag tagged NLRP3 construct after removal of the c-terminal Flag coding sequence. The TFEB-GFP plasmid is bought from Addgene, (38119) and the TFEB-mCherry plasmid made by moving the TFEB cDNA into the mCherry N1 vector (ClonTech). Pooled siRNAs targeting NLRP3 (sc-45469), ASC (sc-37281), GFP (sc-45924), and a scrambled control (sc-37007) are purchased from Santa Cruz Biotechnology. Plasmids and siRNA were transfected into cells using X-tremeGENE-HP (Roche, 6366236001) following the manufacture's protocol.

14/ Immunoblot Analysis

For standard immunoblotting, cells are lysed in Buffer A containing 20 mM HEPES (pH 7.4), 50 mM β-glycerophosphate, 1% (v/v) Triton X-100, 2 mM EGTA, and cOmplete protease inhibitor cocktail (Sigma, 11836170001) plus PhosStop (Sigma, 04906837001) phosphatase inhibitor tablets for 30 min. Lysates are cleared by centrifugation at 14,000 rpm for 10 min and the supernatant collected. For separation of Triton soluble and insoluble fractions, cells are lysed in Buffer A and centrifuged as before. The supernatant was considered the Triton soluble fraction, while the pellet was directly mixed with 1× NuPage LDS Sample Buffer (Invitrogen, NP0008) and heated at 100° C. for 30 min (Triton insoluble fraction). For nuclear fractionation, cells are lysed with Buffer B, which is Buffer A substituting 0.5 (v/v) NP-40 for Triton X-100. After centrifugation, the supernatant was considered the cytosolic fraction. The resultant pellet was washed 4-6 times with Buffer B and then lysed in Buffer B+0.5% (v/v) SDS for 30 min, this was considered the nuclear fraction. Inflammasome activation is measured by immunoblotting cell culture supernatants for mature IL-1β and cleaved caspase-1 as previously described (Shi, C. S. et al. Activation of autophagy by inflammatory signals limits IL-lbeta production by targeting ubiquitinated inflammasomes for destruction. Nat. Immunol. 2012, 13:255-263). All lysates are mixed with 4× NuPage LDS Sample Buffer and heated at 100° C. for 10 min, followed by separation on a 4-20% Tris-Gylcine Gel (Invitrogen) and transfer to a nitrocellulose membrane using the iBlot Gel Transfer System (Invitrogen). The membrane is blocked with 5% nonfat milk (or 5% BSA) in TBST (25 mM Tris-HCl, 150 mM NaCl, 0.1% Tween-20) for 1 h and incubated at 4° C. overnight with the primary Ab in TBST with 5% BSA. The appropriate secondary Abs conjugated to HRP is used to detect the protein of interest via ECL. When necessary, membranes were stripped using Restore Plus Western Blot Stripping Buffer (Thermo, 46430) following the manufacturers protocol, re-blocked, and reblotted. Images were acquired either by exposure on film (Amersham Hyperfilm ECL) or using the iBright 1000FL (Invitrogen). Blots were scanned and quantification of band intensity is performed using standard methods on ImageJ (NIH) and presented relative to the appropriate housekeeping gene.

15/ Immunofluorescence

THP-1 cells transiently transfected to express GFP-8b or GFP are seeded on glass-bottom collagen coated microwell dishes (MatTek), differentiated with PMA, and incubated overnight with LPS (50 ng/ml) and a caspase-1 inhibitor (10 μM). The following day, the cells were washed, fixed with 4% paraformaldehyde, permeabilized with 0.1% Triton X-100 in PBS, and blocked with 1% bovine serum albumin for 15 min. For immunostaining, the cells are incubated for 4 h at room temperature or overnight at 4° C. with primary antibodies diluted in 1% bovine serum albumin/PBS. Fluorescent images are collected after secondary antibody incubation using a Leica TCS-SPS-X-WLL confocal microscope equipped with an argon and white light laser (Leica Microsystems) at either 63× or 100× oil immersion objective (NA 1.4). The samples are excited with 488- or 561-nm laser lines. Image analysis was performed using lmaris 8.0.0 (Bitplane AG) and Adobe Photoshop CS3 (Adobe Systems). HeLa cells transiently transfected to express NLRP3-Ds-Red plus or minus GFP-8b are washed 6 h after transfection and plated in glass-bottom microwell dishes overnight. Plated cells are processed and imaged the following day. For DNA staining, the cells are incubated with 30 nM DAPI (Sigma) in PBS for 30 min prior to imaging.

16/ Cell Death Assay

0.4% Tryptan Blue (Sigma T8154) was added to cell culture plates and incubated at room temperature for 2 min. Percentage of non-viable cells (cells that had taken up Tryptan Blue) is determined manually by light microscopy and recorded; a minimum of three fields with >50 cells per field were quantified.

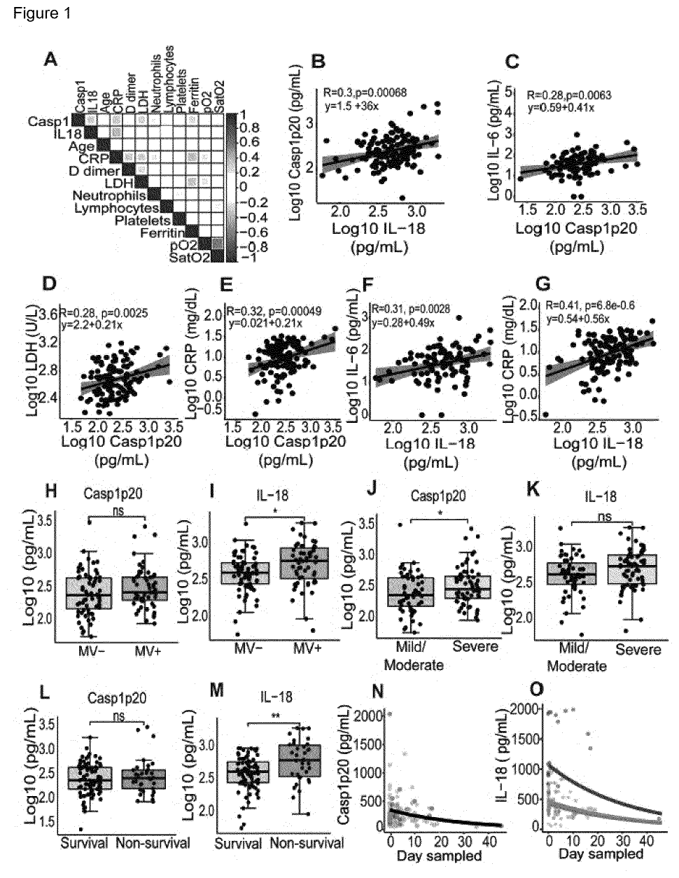

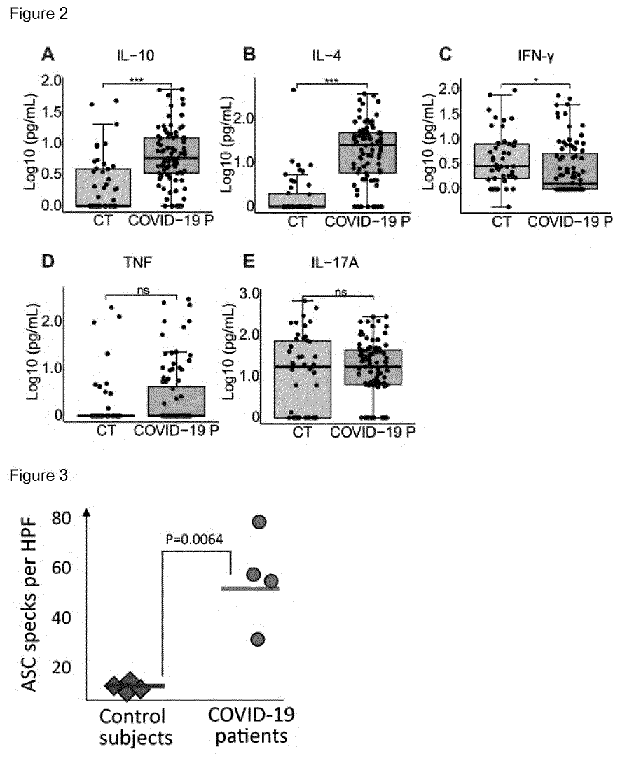

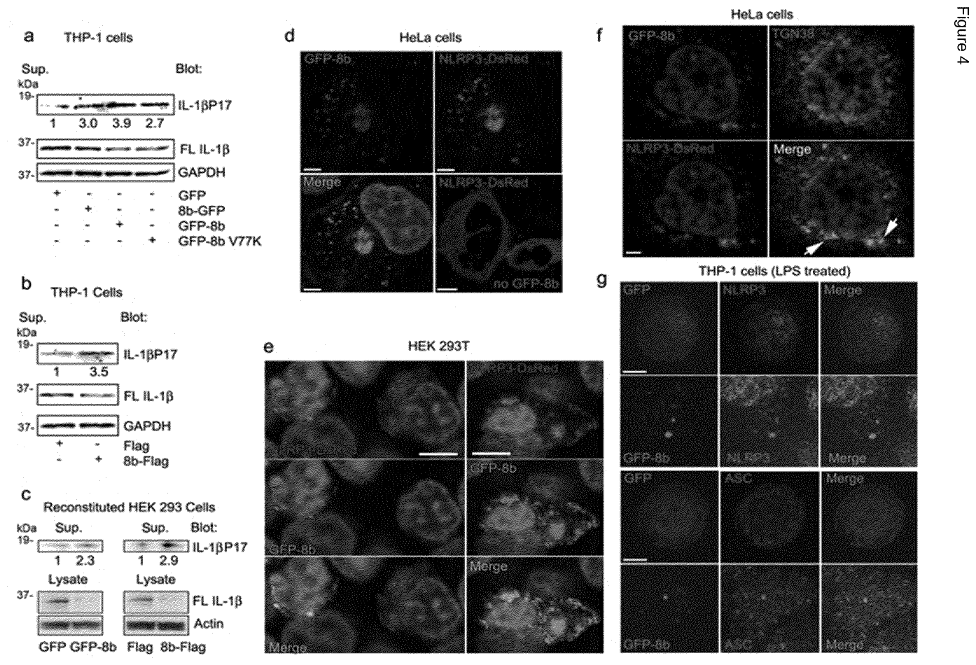

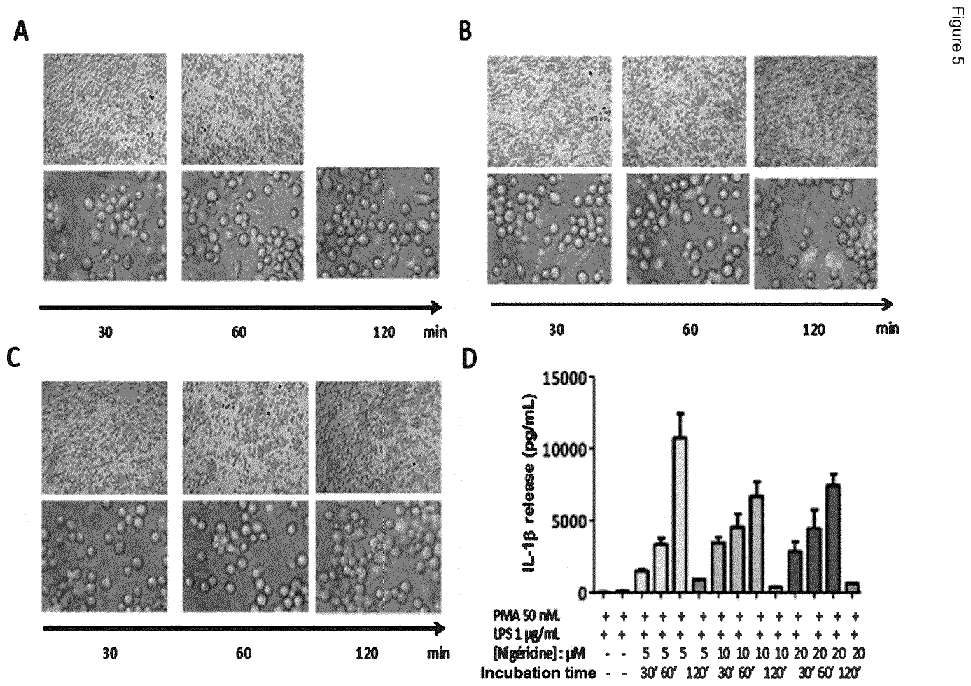

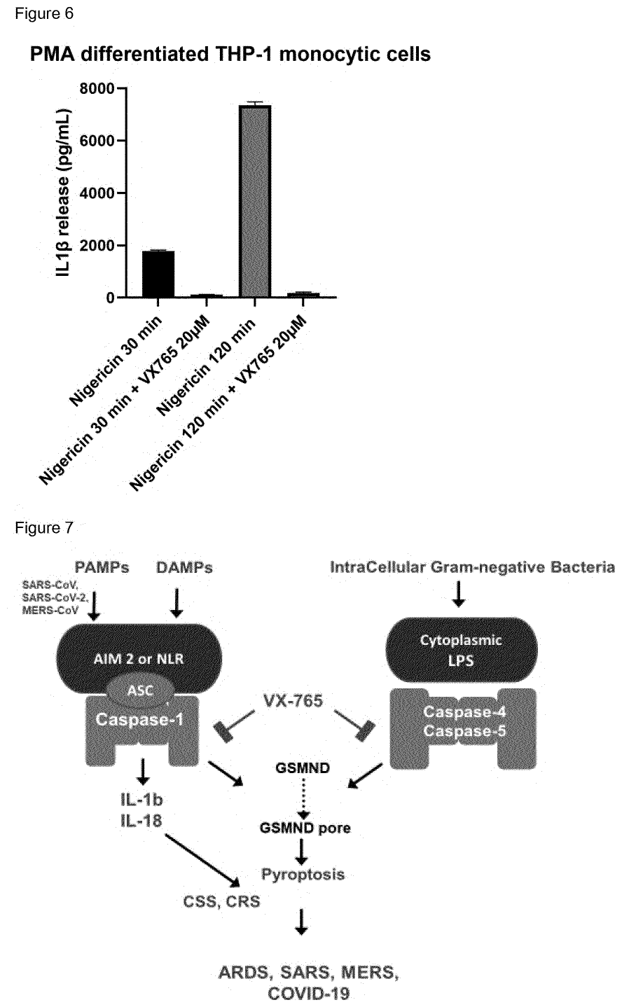

Results are shown in FIGS. 1 to 6.

1. A method for ameliorating, treating, or preventing coronavirus infection, coronavirus disease 2019 (COVID-19), multisystem inflammatory syndrome in children (MIS-C) associated with coronavirus disease 2019 (COVID-19), Severe Acute Respiratory Syndrome (SARS), Middle East Respiratory Syndrome (MERS), pathogen-related respiratory distress syndrome, pathogen-related multi-organ failure, cytokine release syndrome, cytokine storm syndrome, or diseases associated with excessive activation of the canonic and non-canonic inflammasomes, wherein the method comprises comprising administering to a patient in need of such treatment an effective amount of an inhibitor of inflammatory caspases characterized in that it selectively or preferentially inhibits at least one or all the so-called inflammatory Caspases including human Caspase-1, human Caspase-4, and human Caspase-5.

2. A method as defined in claim 1, when the said inhibitor of inflammatory caspases is selected among a group comprising: 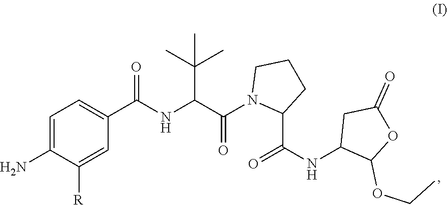
and each stereoisomer thereof, including: 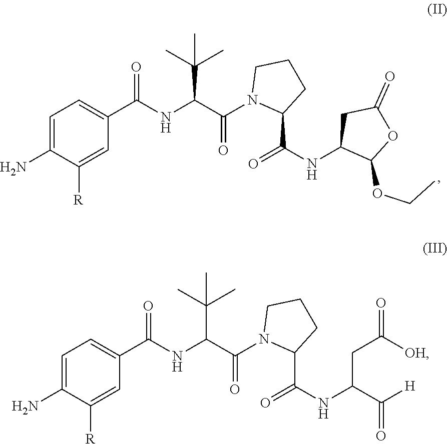
and each stereoisomer thereof, including: 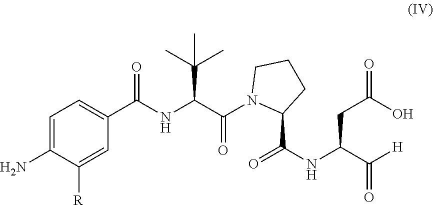
wherein R is H, OH, CH₃, CI, or another halogen, or a pharmaceutical salt, 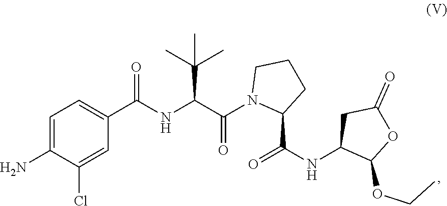 [SMILES: CCO[C@@H]1OC(=O)C[C@@H]1NC(=O)[C@@H]1CCCN1C(=O)[C@@H](NC(=O)c1ccc(N)c(Cl)c1)C(C)(C)C]
also known as Belnacasan or (2S)-1-[(2S)-2-[(4-amino-3-chlorobenzoyl)amino]-3,3-dimethylbutanoyl]-N-[(2 R, 3S)-2-ethoxy-5-oxooxolan-3-yl]pyrrolidine-2-carboxamide,
or a pharmaceutical salt thereof as defined above, 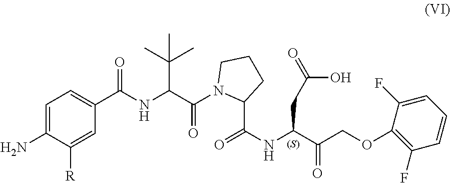
wherein R is H, OH, CH₃ or a halogen, or a pharmaceutical salt, 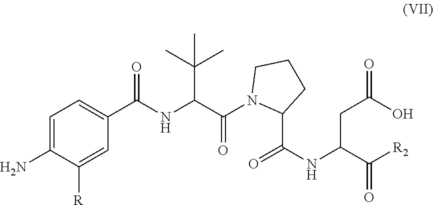
wherein R is H, OH, CH₃ or a halogen, or a pharmaceutical salt, and wherein R2 is 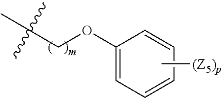
In which m is 0, 1 or 2
Z is a halogen,
p is 1, 2, 3, 4, or 5;
or a pharmaceutically acceptable salt thereof; and 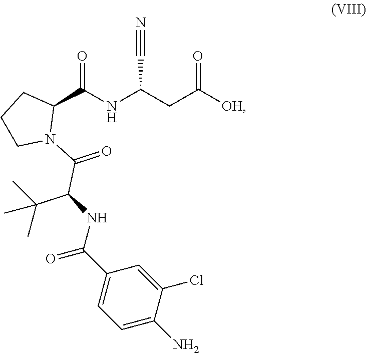 [SMILES: CC(C)(C)[C@H](NC(=O)c1ccc(N)c(Cl)c1)C(=O)N1CCC[C@H]1C(=O)N[C@H](C#N)CC(=O)O]
or a pharmaceutically acceptable salt thereof;
or a pharmaceutical composition containing either entity

3. A method as defined in claim 1, wherein said inhibitor of inflammatory caspases, salt or composition is administered to the patient by oral, parenteral, intravenous, intramuscular, intranasal, sublingual, intratracheal, inhalation, ocular, vaginal, and rectal route.

4. A method as defined in claim 1, comprising administering one or more than one intravenous doses of a therapeutically effective dose of said inhibitor of inflammatory caspases.

5. The method of claim 1, wherein said inhibitor of inflammatory caspases further comprises at least one pharmaceutically acceptable carrier.

6. The method of claim 1, wherein said inhibitor of inflammatory caspases is administered to the patient at a dose of 300 mg to 2,400 mg per administration.

7. The method of claim 6, wherein said inhibitor of inflammatory caspases is administered to the patient at a dose of 600 mg to 1,800 mg per administration.

8. The method of claim 7, wherein said inhibitor of inflammatory caspases is administered to the patient at a dose of 900 mg per administration.

9. The method of claim 1, wherein said inhibitor of inflammatory caspases is administered to the patient at a dose of 2 to 200 mg/kg of body weight/day.

10. The method of claim 9, wherein said inhibitor of inflammatory caspases is administered to the patient at a dose of 6 to 100 mg/kg of body weight/day.

11. The method of claim 10, wherein said inhibitor of inflammatory caspases is administered to the patient at a dose of 25 to 75 mg/kg of body weight/day.

12. The method of claim 1 comprising administering a second active ingredient selected among N-Acetyl-cystein, Fibrin-derived peptide Bβ15-42, Vitamin D, Molnupiravir.

13. The method of claim 1 comprising administering an antibiotic or several antibiotics among ceftriaxone, spiramycin, amoxicillin, amoxicillin/clavulanic acid, gentamicin, netilmicin, piperacilin/tazobactam, am ikacin, cefuroxime, penicillin, azithromycin, clarithromycin, erythromycin, doxycycline, cefotaxime, ampicillin, Ertapenem, cefepime, imipenem, meropenem, metronidazole, fluconazole, ciprofloxacin, levofloxacin, vancomycin, linezolid, moxifloxacin and gem ifloxacin.

14. The method of claim 1, when the patient is infected by a coronavirus or has been admitted to hospital following a PCR detection of a pathogenic coronavirus.

15. The method of claim 1, when the patient is infected by the SARS-CoV-2 or has been admitted to hospital following a PCR detection of SARS-CoV-2.

16. The method defined in claim 1, when the patient is admitted to intensive care unit with elevated markers inflammation, low oxygen levels, or acute respiratory distress.

17. A pharmaceutical composition, wherein the pharmaceutical composition comprises an inhibitor of inflammatory caspases that inhibits selectively or preferentially at least one or all the so-called inflammatory Caspases including human Caspase-1, human Caspase-4, and human Caspase-5, for ameliorating, treating, or preventing coronavirus infection, coronavirus disease 2019 (COVID-19), multisystem inflammatory syndrome in children (MIS-C) associated with coronavirus disease 2019 (COVID-19), Severe Acute Respiratory Syndrome (SARS), Middle East Respiratory Syndrome (MERS), pathogen-related respiratory distress syndrome, pathogen-related multi-organ failure, cytokine release syndrome, cytokine storm syndrome, or diseases associated with excessive activation of the canonic and non-canonic inflammasomes.

18. A pharmaceutical composition according to claim 17, wherein said inhibitor of inflammatory caspases is selected among: 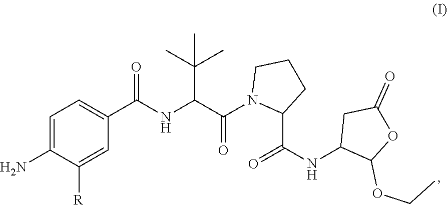
and each stereoisomer thereof, including: 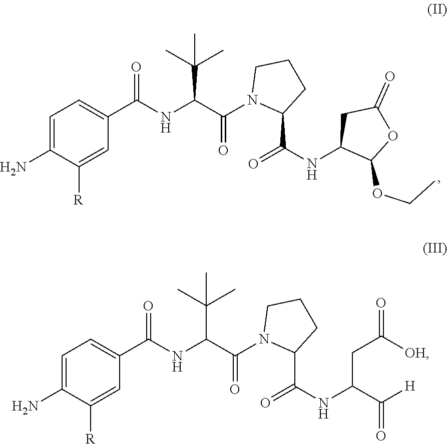
and each stereoisomer thereof, including: 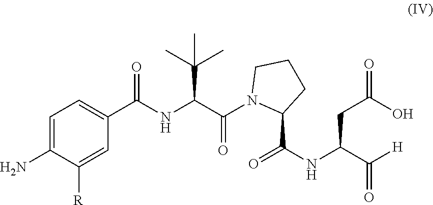
wherein R is H, OH, CH₃, Cl, or another halogen, 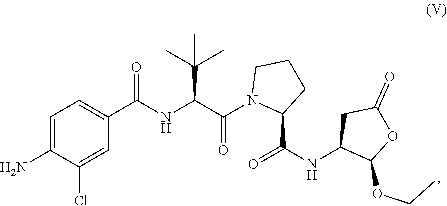 [SMILES: CCO[C@@H]1OC(=O)C[C@@H]1NC(=O)[C@@H]1CCCN1C(=O)[C@@H](NC(=O)c1ccc(N)c(Cl)c1)C(C)(C)C]
also known as Belnacasan or (2S)-1-[(2S)-2-[(4-amino-3-chlorobenzoyl)amino]-3,3-dimethylbutanoyl]-N-[(2R,3S)-2-ethoxy-5-oxooxolan-3-yl]pyrrolidine-2-carboxamide,
or a pharmaceutical salt thereof.

19. A pharmaceutical composition according to claim 17, wherein the inhibitor of inflammatory caspases selected among the group comprising: 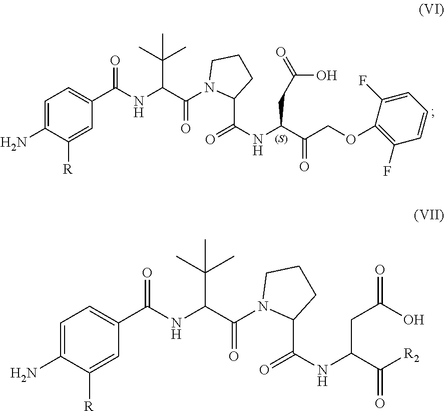
wherein R is H, OH, CH₃ or a halogen and wherein R2 is 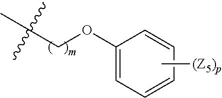
In which m is 0, 1 or 2
Z is a halogen,
p is 1, 2, 3, 4, or 5,
wherein R is H, OH, CH₃ or a halogen, and 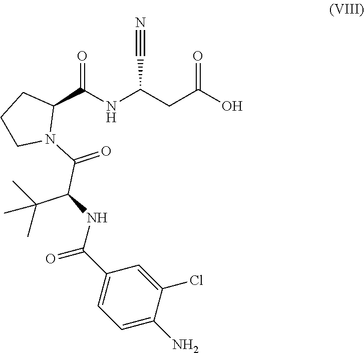 [SMILES: CC(C)(C)[C@H](NC(=O)c1ccc(N)c(Cl)c1)C(=O)N1CCC[C@H]1C(=O)N[C@H](C#N)CC(=O)O]
or a pharmaceutical salt thereof, and each stereoisomer thereof.

20. A pharmaceutical composition according to claim 17 claims 17 to 19, wherein the pharmaceutical composition is administered by oral, intravenous, parenteral, intramuscular, intranasal, sublingual, intratracheal, inhalation, ocular, vaginal, and rectal route.

21. A pharmaceutical composition according to claim 17, wherein the pharmaceutical composition is administered to the patient at a dose of 2 to 200 mg/kg of body weight/day.

22. A pharmaceutical composition according to claim 21, wherein the pharmaceutical composition is administered to the patient at a dose of 6 to 100 mg/kg of body weight/day.

23. A pharmaceutical composition according to claim 22, wherein the pharmaceutical composition is administered to the patient at a dose of 25 to 75 mg/kg of body weight/day.

24. A pharmaceutical composition according to claim 17, further comprising a second active ingredient.

25. The pharmaceutical composition according to claim 24, wherein the second active ingredient is selected among N-Acetyl-cysteine, Fibrin-derived peptide Bβ15-42, Vitamin D, Molnupiravir, and a SARS-CoV-2 protease inhibitor including PF-07321332 and PF-07304814.

26. The pharmaceutical composition according to claim 24, wherein the second active ingredient includes one or several antibiotics among:
ceftriaxone, spiramycin, amoxicillin, amoxicillin/clavulanic acid, gentamicin, netilmicin, piperacilin/tazobactam, amikacin, cefuroxime, penicillin, azithromycin, clarithromycin, erythromycin, doxycycline, cefotaxime, ampicillin, Ertapenem, cefepime, imipenem, meropenem, metronidazole, fluconazole, ciprofloxacin, levofloxacin, vancomycin, linezolid, moxifloxacin and gem ifloxacin.

27. A method for reduction of Caspase-1 activation, Caspase-4 activation, Caspase-5 activation, IL-1β maturation and release, IL-1α release, IL-18 maturation, Pyroptosis, and release or other biomarkers in a patient having coronavirus infection, coronavirus disease 2019 (COVID-19), multisystem inflammatory syndrome in children (MIS-C) associated with coronavirus disease 2019 (COVID-19), Severe Acute Respiratory Syndrome (SARS), Middle East Respiratory Syndrome (MERS), pathogen-related respiratory distress syndrome, pathogen-related multi-organ failure, cytokine release syndrome, cytokine storm syndrome, or diseases associated with excessive activation of the canonic and non-canonic inflammasomes, wherein the method comprises the step of administering to a patient the pharmaceutical composition of claim 17.
